(19) United States
(12) Patent Application Publication
(10) Pub. No.: US 2003/0104385 A1
(43) Pub. Date: Jun. 5, 2003

(54) Nucleic acids and encoded polypeptides associated with bipolar disorder

(76) Inventor: Glen A. Evans, San Marcos, CA (US)

Correspondence Address: CAMPBELL & FLORES LLP, 4370 LA JOLLA VILLAGE DRIVE, 7TH FLOOR, SAN DIEGO, CA, 92122, US

(21) Appl. No.: 09/922,225

(22) Filed: Aug. 2, 2001

(51) Int. Cl. C12Q 1/68; G01N 33/53; A01K 67/00; C12P 21/02; C12N 5/06; C07H 21/04; C12N 9/10; C07K 16/40

(52) U.S. Cl. 435/6; 435/7.1; 435/69.1; 435/193; 435/320.1; 435/325; 536/23.2; 530/388.26; 800/8

(57) ABSTRACT

The present invention provides an isolated human mannosyl transferase and the encoding nucleic acid. Nucleic acids and fragments thereof that correspond to the mannosyl transferase polypeptide similarly are applicable in therapeutic procedures. The invention also provides a human mannosyl transferase fusion polypeptide and a chromosome 9 fusion polypeptide, both of which result from a chromosomal translocation t(9,11) (p24;q23.1). The fusion nucleic acid sequence that encodes the human mannosyl transferase fusion polypeptide and the fusion nucleic acid sequence that encodes the chromosome 9 fusion polypeptide also are provided. The fusion proteins of the invention and their encoding nucleic acids are useful in methods provided by the present invention for diagnosing or predicting the susceptibility to bipolar disorder.

### BACKGROUND OF THE INVENTION

The present invention relates generally to the fields of molecular medicine and biochemistry and, more specifically, to nucleic acids and proteins associated with bipolar disorder.

Manic-depressive illness, known in medical terms as bipolar disorder, is a common illness characterized by recurrent mood episodes of excessive highs (mania) to profound hopelessness (depression), usually with periods of normal mood in between. The type, severity and duration of mood episodes can vary. Some individuals may have a predominance of either mania or depression, whereas other sufferers may experience equal numbers of both. The mood episodes can last for a few days to as long as several months, particularly when left untreated or not treated effectively. Typically, a person with bipolar disorder can expect an average of ten episodes of mania or depression in his or her lifetime but some sufferers experience much more frequent mood episodes. The frequency of episodes tends to increase with time and individuals who experience four or more episodes in a year are said to have rapid cycling bipolar disorder, which affects between 13 to 20 percent of individuals diagnosed with bipolar disorder.

Manic episodes are characterized by euphoria, constant talkativeness or movement, grandiose thoughts, need for less sleep, inability to focus, and reckless behavior. When severe mania sets in, the dividing line between reality and fantasy is crossed. Delusional ideation, paranoia, hallucinations, and disorganized behavior may be seen in full-blown mania, with patients requiring hospitalization to protect both themselves and those around them. Untreated, the manic phase can last as long as three months. As it abates, the patient may have a period of normal mood and behavior. Eventually, however, the depressive phase of the illness will set in. In some sufferers, depression occurs immediately or within the next few months, while for others there is a long interval before the next manic or depressive episode. The depressive phase has the same symptoms as major or unipolar depression, including feelings of worthlessness and hopelessness, inability to concentrate, thoughts of death or suicide, change in appetite or weight, and fatigue or loss of energy.

Approximately 1 to 2 percent of adults suffer from the illness, which affects both men and women. According to the National Depressive and Manic-Depressive Association, bipolar disorder affects 2.5 million adult Americans sometime during their lifetime. Furthermore, with regard to the incident rate of bipolar disorder striking similarities exist between different countries and cultures. While people can be afflicted anytime during their lifetime, a typical age of onset for bipolar disorder is in the range of 18 to 22 years. Available evidence suggests that bipolar disorder runs in families and is inheritable. In this regard, close relatives of people suffering from bipolar illness are 10 to 20 times more likely to develop either depression or manic-depressive illness than the general population.

Two out of three people with mood disorders do not get proper treatment because their symptoms are not properly recognized or misdiagnosed. On average, people with bipolar disorder consult 3.3 doctors over eight years before receiving a correct diagnosis of bipolar disorder. Recognition of the illness, in particular the manic episodes, can often be difficult and bipolar disorder often can be mistaken for unipolar depression.

Bipolar disorder is associated with high levels of service use and morbidity and it has been estimated that approximately 15 percent of patients eventually die of suicide. While lithium remains the first line drug of choice for treatment of bipolar disorder, it does not prevent recurrence in approximately 40 percent of patients. More recently a variety of anticonvulsants have been used prophylactically as mood stabilizers to reduce the recurrence of depression and mania. Overall, the treatments currently available for bipolar disorder have undesirable side effects and lack effectiveness for all patients. In addition, the pathogenesis of bipolar disorder remains poorly understood and diagnosis of the disease remains difficult.

Family, twin, and adoption studies have provided strong evidence for a genetic etiology in bipolar disorder (reviewed in Craddock and Jones, J. Med. Genet. 36:585-594(1999)). Early reports of linkage of bipolar disorder to DNA markers at several chromosomal sites have not proven robust, likely because of the complex nature of bipolar disorder inheritance, which may result from several genes acting in concert. Molecular genetic positional and candidate gene approaches are being used for the genetic dissection of bipolar disorder. The identification of bipolar susceptibility genes will contribute to the understanding of disease pathophysiology and prove important opportunities to investigate the interaction between genetic and environmnetal factors involved in pathogenesis. Moreover, the identification of bipolar susceptibility genes is likely to lead to improvements in treatment and patient care.

Thus, there exists a need to elucidate the genetic basis of bipolar disorder and develop reliable diagnostic markers and effective treatments associated with high response rates. The present invention satisfies this need and provides related advantages as well.

### SUMMARY OF THE INVENTION

The present invention provides an isolated human mannosyl transferase and the encoding nucleic acid. The mannosyl transferase polypeptide of the invention is encoded by a gene that is located on chromosome 11 and disrupted in a chromosomal translocation t(9,11) (p24;q23.1), which cosegregates with bipolar disorder. The mannosyl transferase polypeptide and functional fragments thereof are useful in therapeutic procedures for bipolar disorder. Nucleic acids and fragments thereof that correspond to the mannosyl transferase polypeptide similarly are applicable in therapeutic procedures.

The invention also provides a human mannosyl transferase fusion polypeptide and a chromosome 9 fusion polypeptide, both of which result from the chromosomal translocation t(9,11) (p24;q23.1). The fusion nucleic acid sequence that encodes the human mannosyl transferase fusion polypeptide and the fusion nucleic acid sequence that encodes the chromosome 9 fusion polypeptide also are provided. The fusion proteins of the invention and their encoding nucleic acids are useful in methods provided by the present invention for diagnosing or predicting the susceptibility to bipolar disorder.

### BRIEF DESCRIPTION OF THE DRAWINGS

FIG. 1 shows a partial pedigree of a family with cosegregation of a balanced t(9;11) translocation and bipolar disorder (NL, normal karyotype, 9;11, carrier of balanced translocation).

FIG. 2 shows the (A) cDNA sequence of the human mannosyl transferase protein interrupted by the t(9; 11) breakpoint and (B) the corresponding amino acid sequence.

FIG. 3 shows an (A) open reading frame having a 5′ coding sequence from a gene located on chromosome 9 and a 3′ coding sequence corresponding to a human mannosyl transferase gene located on chromosome 11, which is created by the insertion of a chromosome 9 sequence into chromosome 11, and (B) a schematic of the corresponding fusion protein with a 5′ portion translation product of chromosome 9 sequence and a 3′ portion translation product of chromosome 11 sequence.

FIG. 4 shows the 5′ fusion transcript and corresponding amino acid sequence corresponding to the mannosyl transferase fusion polypeptide.

FIG. 5 shows the chromosome 9 fusion, or 11/9 fusion, having a 5′ coding sequence corresponding to a human mannosyl transferase gene located on chromosome 11 and a 3′ coding sequence corresponding to a gene located on chromosome 9, which is created by the insertion of a chromosome 11 sequence into chromosome 9.

FIG. 6 shows an alternative mannosyl transferase fusion, or 9/11 fusion, having a 5′ coding sequence corresponding to a gene located on chromosome 9 and a 3′ coding sequence corresponding corresponding to a human mannosyl transferase gene located on chromosome 11, which is created by the insertion of a chromosome 9 sequence into chromosome 11.

FIG. 7 shows the chromosome 9 nucleic acid (SEQ ID NO: 9) and amino acid sequence (SEQ ID NO: 10) involved in the gene fusion.

FIG. 8 shows the amino acid sequence of the mannosyl transferase of the invention (SEQ ID NO: 2) and that of the S. cerevisiae (SEQ ID NO: 1l), A. thalania (SEQ ID NO: 12), C. elegans (SEQ ID NO: 13), D. melanogaster (SEQ ID NO: 14), and S. pombe (SEQ ID NO: 15) orthologs and provides percentage sequence identity compared to the human reference mannosyl transferase of the invention (SEQ ID NO: 2).

FIG. 9 shows (A) a schematic of the enzymatic depletion/re-amplification strategy used to determine the upstream 5′ sequence of the chromosome 9;11 translocation breakpoint and (B) amplification of a 490 kb fragment corresponding to the t (9;11) mannosyl transferase fusion indicating amplification of the chromosomal breakpoint, and (C) a schematic of the mannosyl transferase fusion nucleotide and corresponding amino acid dequence.

FIG. 10 shows expression of native and fusion transcripts in normal and t(9;11) cell lines by (A) Northern Blot analysis of RNA isolated from (A) a variety of normal tissues and (B) a cell line carrying the t(9;11) derivative chromosome (T, translocation cell line; C, control cell line).

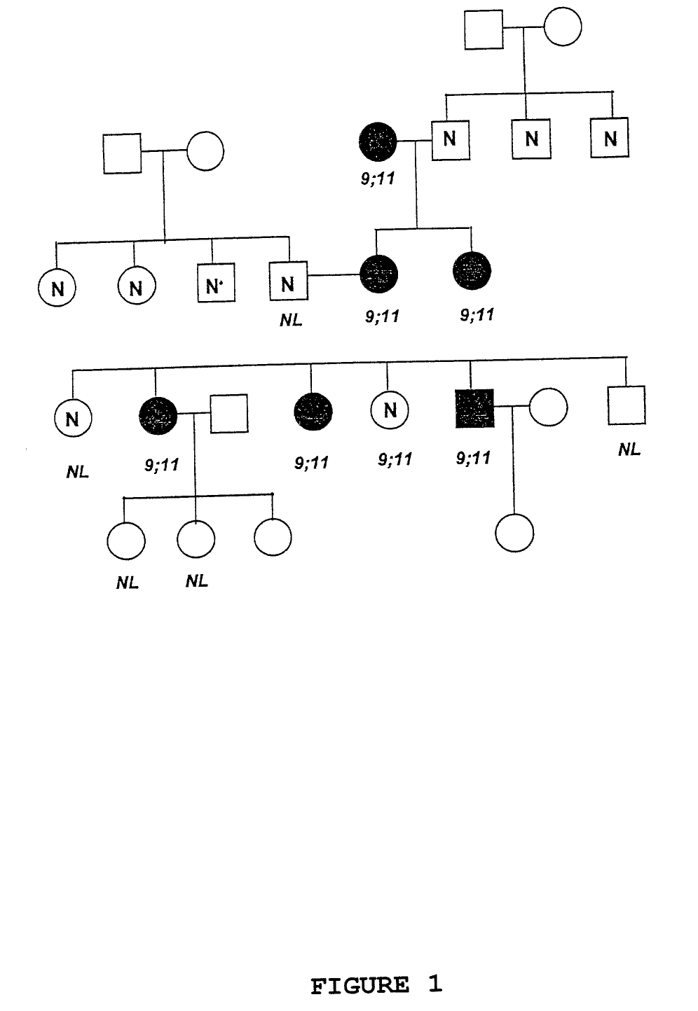

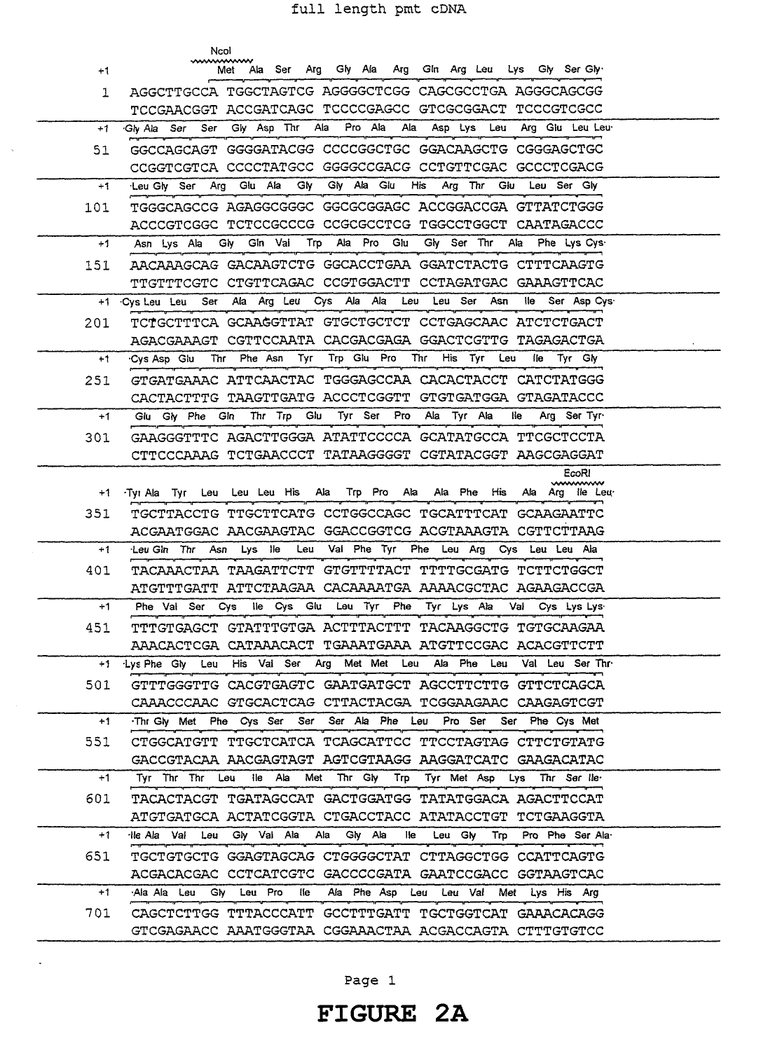

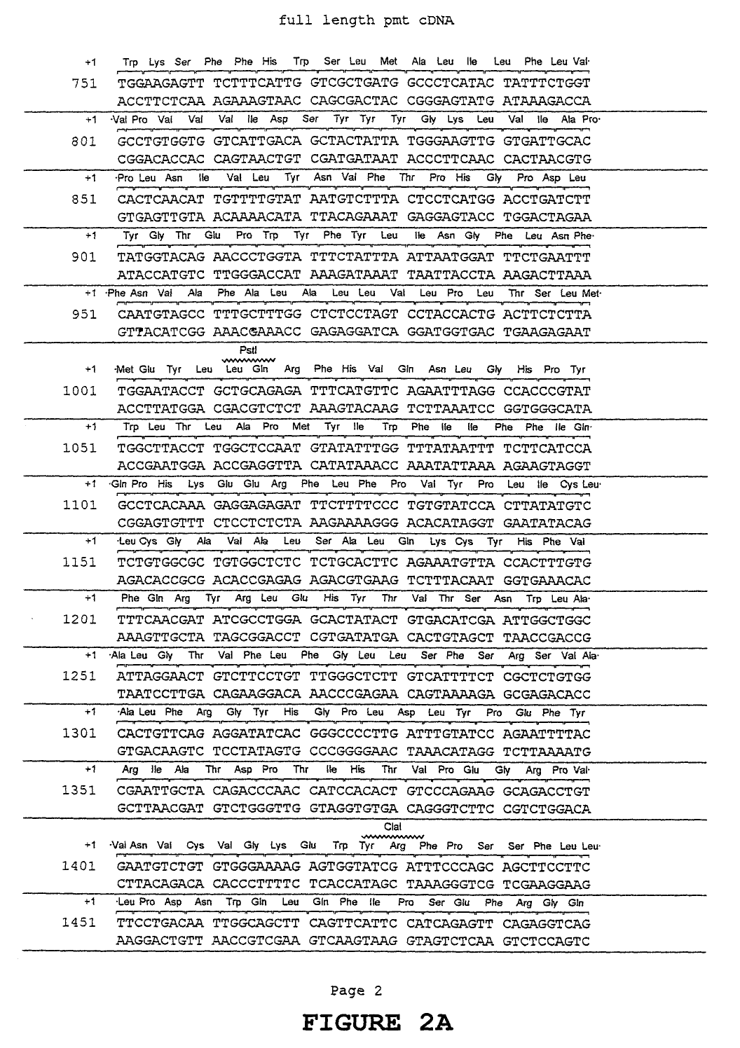

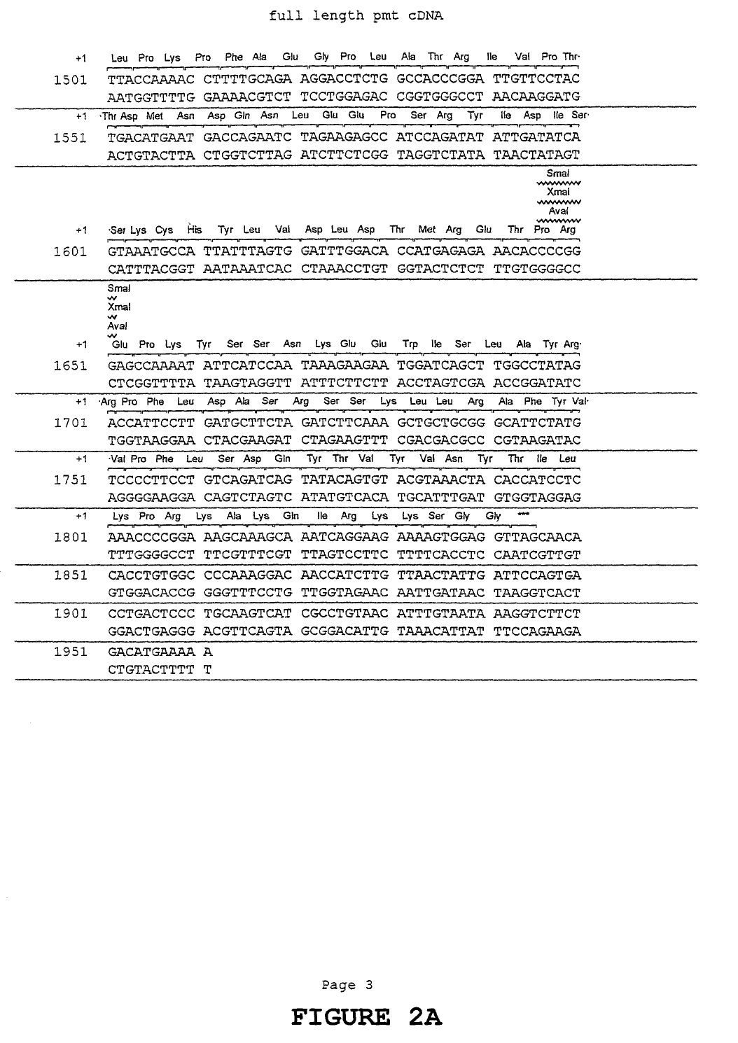

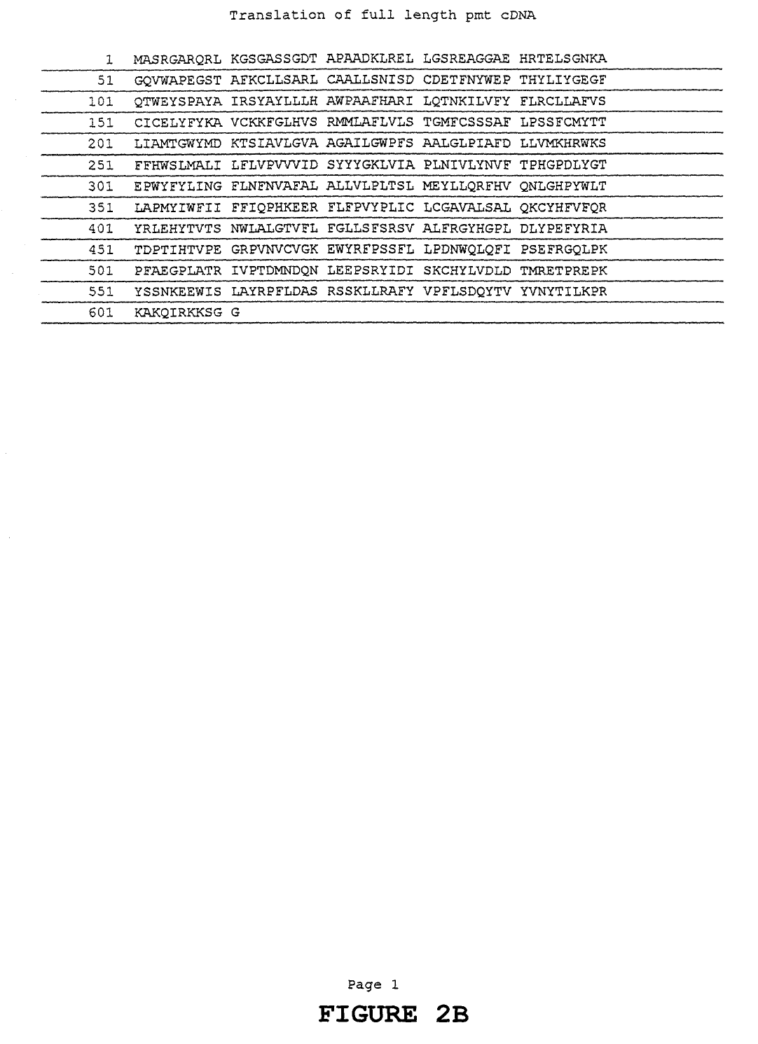

### DETAILED DESCRIPTION OF THE INVENTION

This invention is directed to a mannosyl transferase and encoding nucleic acid. The mannosyl transferase is useful in therapeutic procedures for bipolar disorder. Nucleic acids corresponding to the mannosyl transferase similarly are applicable in therapeutic procedures. The invention also is directed to two fusion polypeptides that result from the chromosomal translocation t(9,11) (p24;q23.1), which cosegregates with bipolar disorder. Nucleic acids corresponding to the fusion polypeptides of the invention also are provided by the present invention. In addition, methods of diagnosing or predicting the susceptibility to bipolar disorder are provided by the invention.

Proteins destined for secretion, incorporation into membranes, or localization inside membranous organelles contain carbohydrates and are therefore classified as glycoproteins. Glycosylation and oligosaccharide processing play an indispensible role in the sorting and the distribution of these proteins to their proper cellular destinations. Three oligosaccharide portions of glycoproteins exist: (1) N-linked oligosaccharides; (2) O-linked oligosaccharides; and (3) Glycosylphosphatidylinositol(GPI)-membrane anchors.

N-linked glycosylation is a modification and follows a conserved pathway in eukaryotic cells (Kornfeld and Kornfeld, Annu. Rev. Biochem. 54:631-664 (1985); Tanner and Lehle, Biochim. Biopphys. Acta 906:81-99 (1987); Herscovics and Orlean, FASEB J. 7:540-550 (1993)). The core oligosaccharide Glc₃Man₉GlcNAc₂ is assembled on the lipid carrier dolichyl pyrophosphate and transferred to selected asparagine residues of nascent polypeptide chains. This transfer is catalyzed by the enzyme oligosaccharyl transferase (Kaplan et al., Biochim. Biopphys. Acta 906:161-173 (1987); Kukuruzinska et al., Annu. Rev. Biochem. 56:914-944). The assembly of lipid linked core oligosaccharide occurs at the membrane of the endoplasmatic reticulum in a stepwise manner (Kornfeld and Kornfeld, supra, 1985; Tanner and Lehle, supra, 1987; Herscovics and Orlean, supra, 1993; Kukuruzinska et al., supra, 1987). The first sugars, two GlcNAc and five Man residues, derive from UDP-GlcNAc and GDP-Man, respectively, whereas the next seven sugars, four Man and three Glc residues, are provided by the lipid intermediates dolichol (Dol)-P-Man and Dol-P-Glc, respectively. Each transfer reaction is mediated by an individual gycosyltransferase (Kornfeld and Kornfeld, supra, 1985; Tanner and Lehle, supra, 1987; Herscovics and Orlean, supra, 1993; Kukuruzinska et al., supra, 1987). In the yeast Saccharomyces cerevisiae, alg (defective in asparagine linked glycosylation) mutations affect different steps in the biosynthesis of the lipid linked core oligosaccharide (Huffaker and Robbins, Proc. Natl. Acad. Sci. USA 80:7466-7470 (1983); Runge et al. J. Biol. Chem. 259:412-417 (1984); Runge and Robbins, J. Biol. Chem. 261:15582-15590 (1986)). These mutations lead to accumulation of lipid-linked sugar intermediates and a reduction of N-linked glycosylation of proteins.

In eukaryotes, the stepwise biosynthesis of lipid-linked oligosaccharides occurs on the lipid carrier dolichylpyrophosphate (Kornfeld and Kornfeld, supra, 1985). In particular, four dolichylphosphomannose-derived mannose residues are added in the lumen of the endoplasmatic reticulum to the lipid linked Man₅GlcNAc₂ oligosaccharide. The first of these mannose residues is added via an α-1,3 linkage to lipid-linked Man₅GlcNAc₂ (Rearick et al., J. Biol. Chem. 256:3762-3769 (1981); Sharma et al., Biochemistry 29:8901-8907 (1990); Verostek et al., J. Biol. Chem. 266:5547-5551 (1991)). While the alg3 mutation in yeast affects the first mannose addition in the lumen of the endoplasmatic reticulum, addition of the next mannose residue to the Man₆GlcNAc₂ intermediate is alg9-dependent.

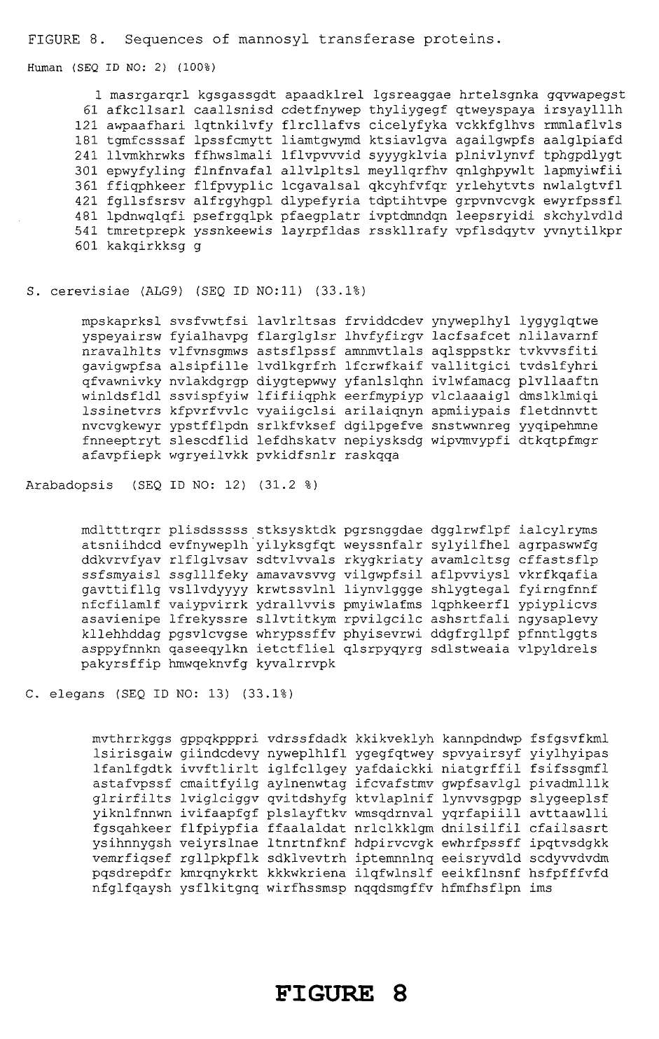

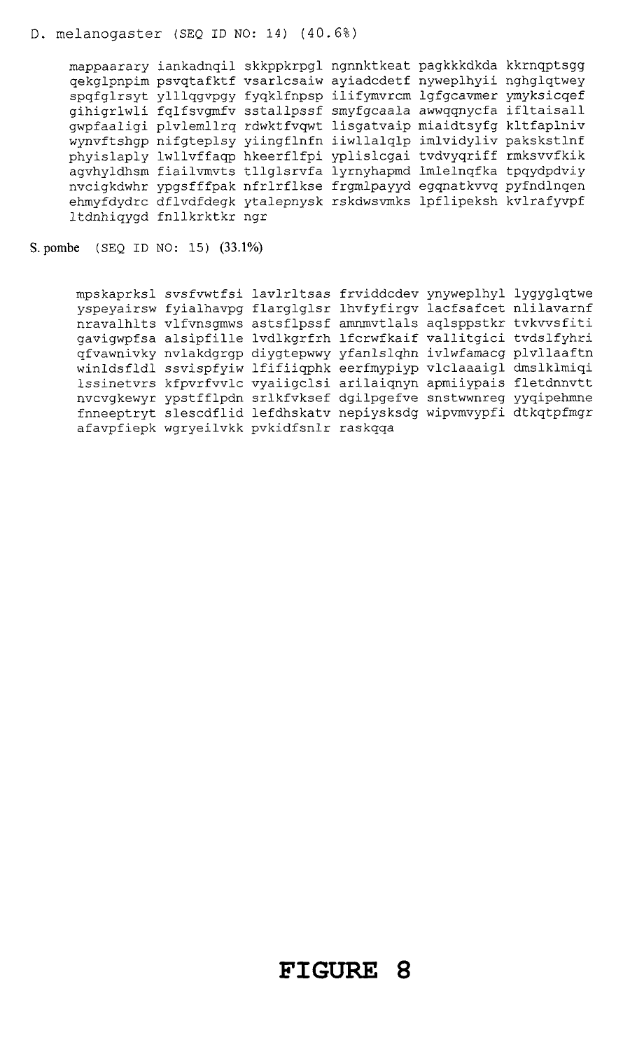

The invention provides an isolated polypeptide that constitutes a mannosyl transferase having substantially the same amino acid sequence as shown as in SEQ ID NO: 2, or a functional fragment thereof. The isolated polypeptide of the invention designated SEQ ID NO: 2 has 33.1 percent sequence identity with the alg9 polypeptide of S. cerevisiae. As shown in FIG. 8, sequence identities between the mannosyl transferase of the invention and homologues that have been identified in other species are as follows: 40.6 percent with D. melanogaster, 33.1 percent with S. pombe, 33.1 percent with C. elegans, 31.2 percent with A. thalania. The mannosyl transferase of the invention can be a human mannosyl transferase. The mannosyl transferase of the invention contains nine transmembrane regions and is located in the endoplasmatic reticulum membrane of cells, where it catalyzes the transfer of mannose from GDP-mannose to the lipid linked oligosaccharide. Furthermore, the isolated mannosyl transferase polypeptide of the invention designated SEQ ID NO: 2 is encoded by a gene of at least 40 kb that is located on chromosome 11 and that is disrupted in a chromosomal translocation t(9,11) (p24;q23.1) that cosegregates with bipolar disorder. Thus, the invention also provides an isolated nucleic acid corresponding to a genomic sequence of approximately 40 kb located on chromosome 11 that encompasses a gene corresponding to the mannosyl transferase of the invention. The nucleic acid sequence corresponding to the mannosyl transferase gene or functional fragment thereof also can contain one or more of the single nucleotide polymorphisms designated SEQ ID NOS: 16-117.

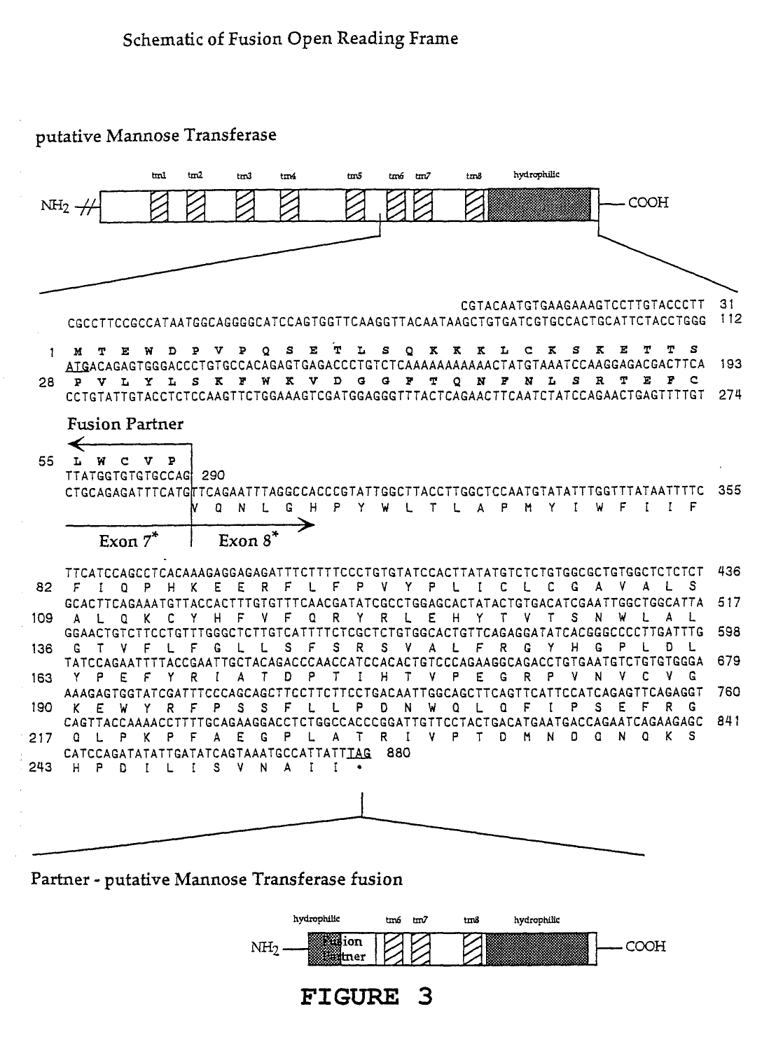

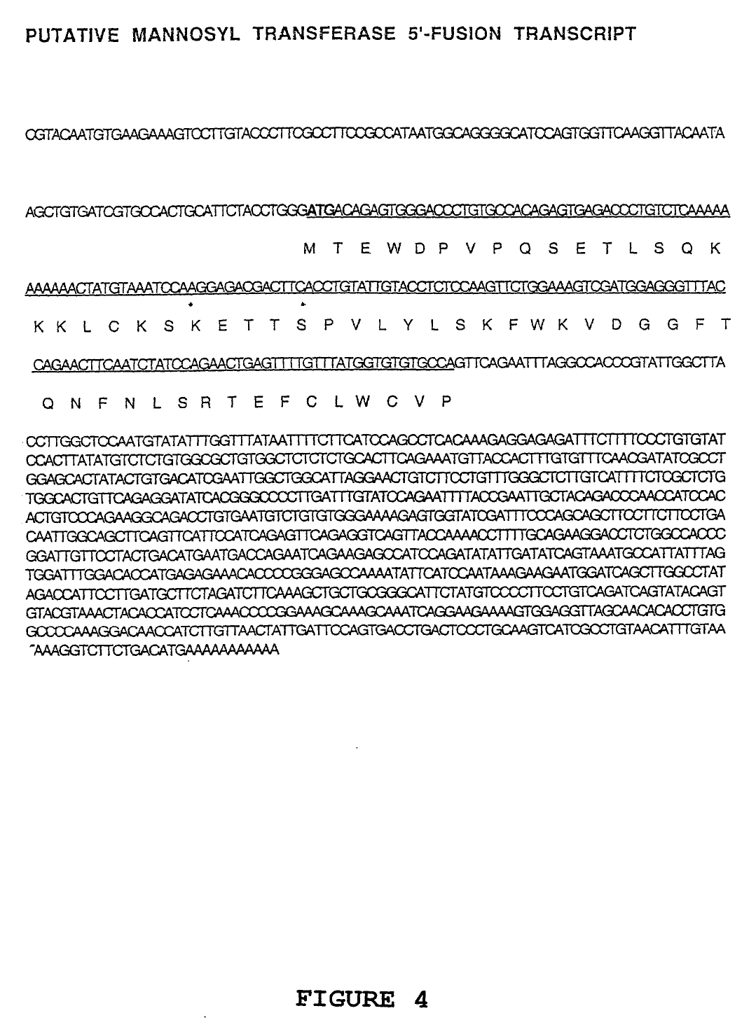

The isolated mannosyl transferase polypeptide of the invention designated SEQ ID NO: 2 is encoded by a gene that is located on chromosome 11 and disrupted in a chromosomal translocation t(9,11) (p24;q23.1), which cosegregates with bipolar disorder. The translocation t(9,11) (p24;q23.1) results in a mannosyl transferase fusion polypeptide, also referred to herein as the 9/11 fusion polypeptide, designated SEQ ID NO: 4. Thus, the invention also provides a polypeptide having substantially the amino acid sequence shown as SEQ ID NO: 4, or a functional fragment thereof. As shown in FIG. 3, a functional fragment of the mannose transferase fusion designated SEQ ID NO: 4, can consist of, for example, amino acid residues 1 to 59, which are encoded by the mannosyl transferase 5′ fusion transcript shown in FIG. 4. The invention also provides an alternate 9/11 fusion polypeptide having substantially the amino acid sequence shown as SEQ ID NO: 6, or a functional fragment thereof.

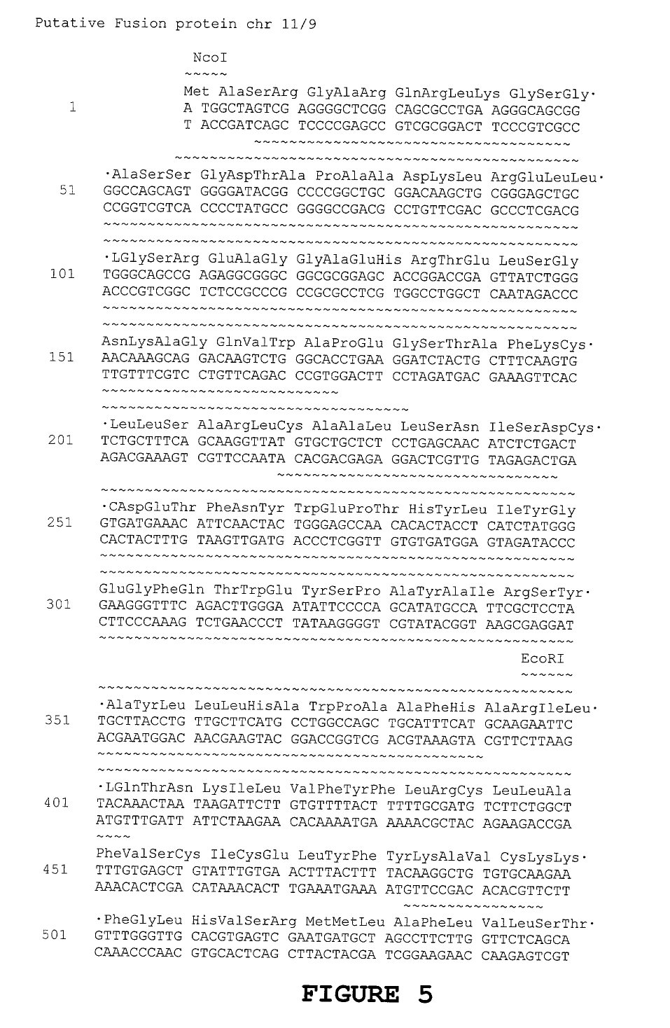

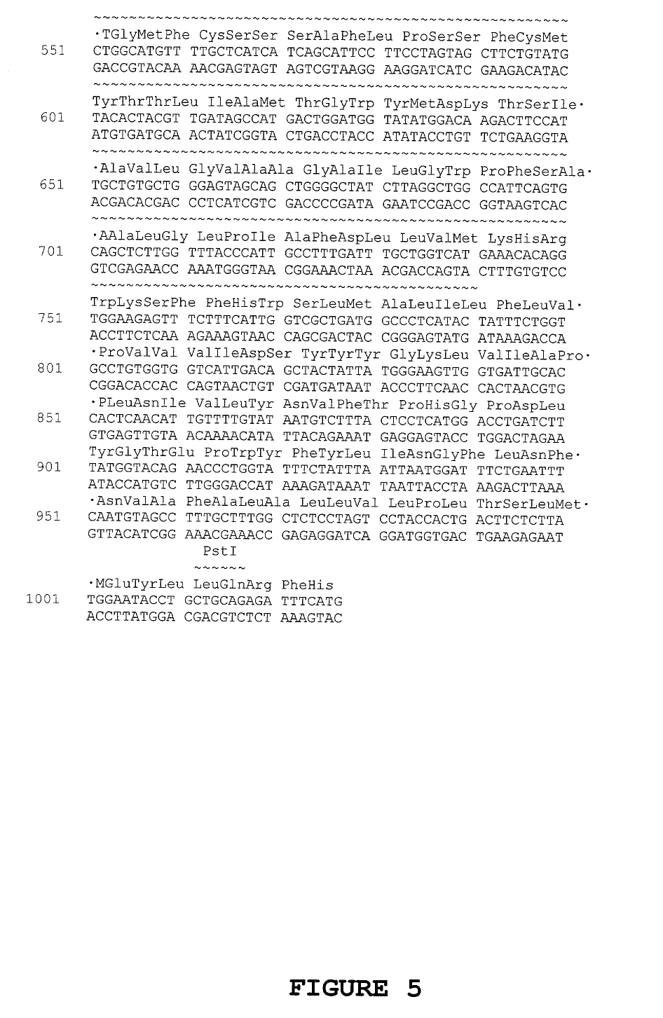

The translocation t(9,11) (p24;q23.1) further results in a chromosome 9 fusion polypeptide, also referred to herein as the 11/9 fusion polypeptide, designated SEQ ID NO: 8. Thus, the invention also provides a polypeptide having substantially the amino acid sequence shown as SEQ ID NO: 8, or a functional fragment thereof. As shown in FIG. 5, a functional fragment of the chromosome 9 fusion polypeptide designated SEQ ID NO: 8, can consist of, for example, of the 3′ amino acid residues encoded by the chromosome 11 region that translocates to chromosome 9.

As used herein, the term “mannosyl transferase” refers to polypeptides having substantially the same amino acid sequence as shown as in SEQ ID NO: 2, or a functional fragment thereof.

As used herein, the term “polypeptide” is intended to mean two or more amino acids covalently bonded together. A polypeptide of the invention includes small polypeptides having a few or several amino acids as well as large polypeptides having several hundred or more amino acids. Usually, the covalent bond between the two or more amino acid residues is an amide bond. However, the amino acids can be joined together by various other means known to those skilled in the peptide and chemical arts. Therefore, a polypeptide, in whole or in part, can include molecules which contain non-amide linkages between amino acids, amino acid analogs, and mimetics. Similarly, the term also includes cyclic peptides and other conformationally constrained structures. A polypeptide also can be modified by naturally occurring modifications such as post-translational modifications, including phosphorylation, lipidation, prenylation, sulfation, hydroxylation, acetylation, addition of carbohydrate, addition of prosthetic groups or cofactors, formation of disulfide bonds, proteolysis, assembly into macromolecular complexes, and the like.

As used herein, the term “substantially the same” when used in reference to an amino acid sequence, refers to an amino acid sequence having at least about 40% identity with the reference amino acid sequence, and retaining comparable functional and biological activity of the polypeptide defined by the reference amino acid sequence. A polypeptide having substantially the same amino acid sequence can have at least about 45%, 50%, 55%, 60%, 65%, 70%, 75%, 80%, 82%, 84%, 86% or 88%, or at least 90%, 91%, 92%, 93% or 94% amino acid identity with respect to the reference amino acid sequence; as well as greater than 95%, 96%, 97%, 98% or 99% amino acid identity. It is recognized, however, that polypeptides, or encoding nucleic acids, containing less than the described levels of sequence identity arising as splice variants or that are modified by conservative amino acid substitutions, or by substitution of degenerate codons also are encompassed within the scope of the present invention.

The term “functional fragment,” when used in reference to an isolated mannosyl transferase polypeptide of the invention, refers to a polypeptide fragment that is a portion of a mannosyl transferase polypeptide or a fusion polypeptide of the invention, provided that the portion has a biological activity, as described herein, that is characteristic of the corresponding isolated mannosyl transferase polypeptide. A functional fragment of the human mannosyl transferase set forth as SEQ ID NO: 2 can be, for example, a substrate binding domain capable of binding a lipid-linked oligosaccharide, a monosaccharide substrate, or both. A polypeptide of the invention or functional fragment of a polypeptide of the invention can exclude those amino acid sequences that were in the public domain as of the filing date of this application, including the specific amino acid sequences available under GenBank Accessions AAH09255, XP_{—}041331, BAB15154, and CAB66861.

In addition, a functional fragment of a mannosyl transferase polypeptide or a fusion polypeptide of the invention can encompass a substrate binding domain. A functional fragment of a mannosyl transferase polypeptide can contain active site residues important for the catalytic activity of the enzyme. Regions important for the glycosylation activity of a mannosyl transferase can be determined or predicted through a variety of methods known in the art. Related proteins such as, for example, yeast oligosaccharyl transferases including alg3 and alg9, phosphoinositol glycan, putative glycolipid alpha mannosyl transferase, GPI anchor biosynthesis protein and dolichol-phosphate-mannose glycolipid, that share a high degree of sequence similarity and can have biochemically similar catalytic properties can provide information regarding the regions important for catalytic activity of a mannosyl transferase. For example, structural modeling can reveal the active site of an enzyme, which is a three-dimensional structure such as a cleft, gorge or crevice formed by amino acid residues generally located apart from each other in primary structure. Therefore, a functional fragment of a mannosyl transferase polypeptide of the invention can encompass amino acid residues that make up regions of a mannosyl transferase important for glycosylation activity such as those residues located along the active site gorge.

In addition to structural modeling of an isolated mannosyl transferase polypeptide or a fusion polypeptide of the invention, biochemical data can be used to determine or predict regions of a mannosyl transferase important for glycosylation activity when preparing a functional fragment of a mannosyl transferase polypeptide of the invention. In this regard, the characterization of naturally occurring oligosaccharyl transferases with altered glycosylation activity can be useful for identifying regions important for the catalytic activity of a mannosyl transferase polypeptide. In addition, the preparation and testing for catalytic activity of deletion mutants of a mannosyl transferase polypeptide of the invention also can be used to determine or predict catalytically important amino acid residues. Similarly, site-directed mutagenesis studies can provide data regarding catalytically important amino acid residues as reviewed, for example, in Schwartz et al., Pharmac. Ther. 67: 283-322 (1992), which is incorporated by reference.

An isolated polypeptide having substantially the amino acid sequence of SEQ ID NOS: 2, 4, 6 or 8, or a functional fragment thereof, can have conservative amino acid substitutions as compared with the mannosyl transferase polypeptide amino acid sequence. Conservative substitutions of encoded amino acids include, for example, amino acids that belong within the following groups: (1) non-polar amino acids (Gly, Ala, Val, Leu, and Ile); (2) polar neutral amino acids (Cys, Met, Ser, Thr, Asn, and Gln); (3) polar acidic amino acids (Asp and Glu); (4) polar basic amino acids (Lys, Arg and His); and (5) aromatic amino acids (Phe, Trp, Tyr, and His).

An isolated polypeptide having substantially the amino acid sequence of SEQ ID NOS: 2, or a functional fragment thereof, also can be chemically modified, provided that the polypeptide retains a biological activity of mannosyl transferase. An isolated polypeptide having substantially the amino acid sequence of SEQ ID NOS: 4, 6 or 8, or a functional fragment thereof, also can be chemically modified, provided that the polypeptide retains a biological activity of the parent polypeptide. For example, chemical modification of a mannosyl transferase polypeptide of the invention can include alkylation, acylation, carbamylation and iodination. For example, modified polypeptides also can include those polypeptides in which free amino groups have been derivatized to form amine hydrochlorides, p-toluene sulfonyl groups, carbobenzoxy groups, t-butyloxycarbonyl groups, chloroacetyl groups or formyl groups. Free carboxyl groups can be modified to form salts, methyl and ethyl esters or other types of esters or hydrazides. Free hydroxyl groups can be modified to form O-acyl or O-alkyl derivatives. The imidazole nitrogen of histidine can be derivatized to form N-im-benzylhistidine. A mannosyl transferase polypeptide or a fusion polypeptide of the invention also can include a variety of other modifications well known to those skilled in the art, provided the biological activity of the mannosyl transferase polypeptide remains substantially unaffected.

An isolated polypeptide having substantially the amino acid sequence of SEQ ID NOS: 2, 4, 6 or 8, or a functional fragment thereof, also can be substituted with one or more amino acid analogs of the twenty standard amino acids, for example, 4-hydroxyproline, 5-hydroxylysine, 3-methylhistidine, homoserine, ornithine or carboxyglutamate, and can include amino acids that are not linked by peptide bonds.

An isolated polypeptide having substantially the amino acid sequence of SEQ ID NOS: 2, 4, 6 or 8, or a functional fragment thereof, also can contain mimetic portions that orient functional groups, which provide a function of a mannosyl transferase polypeptide. Therefore, mimetics encompass chemicals containing chemical moieties that mimic the function of the polypeptide. For example, if a polypeptide contains similarly charged chemical moieties having similar functional activity, a mimetic places similar charged chemical moieties in a similar spatial orientation and constrained structure so that the chemical function of the charged moieties is maintained. Exemplary mimetics are peptidomimetics, peptoids, or other peptide-like polymers such as poly-β-amino acids, and also non-polymeric compounds upon which functional groups that mimic a peptide are positioned.

The invention also provides a chimeric polypeptide encompassing a mannosyl transferase polypeptide of about 611 amino acid residues, that have a first amino acid sequence corresponding to a mannosyl transferase having substantially the same amino acid sequence as shown as SEQ ID NO: 2, or a functional fragment thereof, and a second amino acid sequence corresponding to a non-human mannosyl transferase. The second amino acid sequence of a chimeric protein of the invention corresponds to a non-human mannosyl transferase and can encompass, for example, an amino terminal region or a carboxyl terminal region of such a non-human mannosyl transferase. The second amino acid sequence can correspond, for example, to a yeast mannosyl transferase or to a mammalian mannosyl transferase such as a mouse, primates, including monkey and baboon, rat, rabbit, bovine, porcine, ovine, canine, feline, or other mammalian mannosyl transferase.

A functional fragment of a first amino acid sequence corresponding to a mannosyl transferase can encompass a substrate binding domain and can have substantially the same amino acid sequence as shown as in SEQ ID NOS: 2, or a functional fragment thereof.

A chimeric protein of the invention can be generated, for example, by recombinantly expressing a functional fragment of a mannosyl transferase having substantially the same amino acid sequence as shown as in SEQ ID NO: 2, such as the substrate binding domain fused to a non-human mannosyl transferase polypeptide. Alternatively, the mannosyl transferase polypeptide having substantially the same amino acid sequence as shown as in SEQ ID NO: 2 can be expressed as a fusion to another polypeptide.

A chimeric polypeptide of the invention or functional fragment thereof also can include, for example, luciferase, green fluorescent protein, an antibody, or other proteins or functional fragments thereof that are not derived from mannosyl transferase and that facilitate identification of the chimera. Still further amino acid sequences encompassed in the chimeric polypeptide of the invention or functional fragment thereof will include, for example, the LexA DNA binding domain, ricin, α-sarcin, an antibody, or other proteins which have therapeutic properties or other biological activity.

Since a chimeric polypeptide of the invention includes sequences from at least two different proteins, a human and a non-human mannosyl transferase, the resultant amino acid sequence of the chimeric protein will typically be a non-naturally occurring sequence. Thus, in accordance with this embodiment of the invention, there is provided a chimeric protein encompassing a first amino acid sequence corresponding to a mannosyl transferase having substantially the same amino acid sequence as shown as SEQ ID NOS: 2, or a functional fragment thereof, and a second amino acid sequence corresponding to a non-human mannosyl transferase.

The amino acid length of functional fragments of a mannosyl transferase polypeptide of the present invention can range from about 5 amino acids up to the full-length protein sequence of an invention human mannosyl polypeptide. In certain embodiments, the amino acid lengths include, for example, at least about 10 amino acids, at least about 15, at least about 20, at least about 25, at least about 30, at least about 35, at least about 40, at least about 45, at least about 50, at least about 75, at least about 100, at least about 150, at least about 200, at least about 250 or more amino acids in length up to the full-length mannosyl transferase polypeptide sequence. The functional fragments can be contiguous amino acid sequences of a mannosyl transferase polypeptide, including contiguous amino acid sequence corresponding to the substrate binding domain of the mannosyl transferase polypeptide.

The invention provides isolated human mannosyl polypeptides and functional fragments thereof. A human mannosyl polypeptide can be isolated by a variety of methods well-known in the art, for example, recombinant expression systems described herein, precipitation, gel filtration, ion-exchange, reverse-phase and affinity chromatography, and the like. Other well-known methods are described in Deutscher et al., Guide to Protein Purification: Methods in Enzymology Vol. 182, (Academic Press, (1990)). Alternatively, the isolated polypeptides of the present invention can be obtained using well-known recombinant methods (see, for example, Sambrook et al., Molecular Cloning: A Laboratory Manual, 2nd ed., Cold Spring Harbor Press, Plainview, N.Y. (1989); and Ausubel et al., Current Protocols in Molecular Biology, John Wiley & Sons, New York (2000)). The methods and conditions for biochemical purification of a polypeptide of the invention can be chosen by those skilled in the art, and purification monitored, for example, by an immunological assay or a functional assay.

An example of the means for preparing a polypeptide of the invention is to express nucleic acids encoding mannosyl transferase in a suitable host cell, such as a bacterial cell, a yeast cell, an amphibian cell such as an oocyte, or a mammalian cell, using methods well known in the art, and recovering the expressed polypeptide, again using well-known purification methods, so described herein. Computer directed gene synthesis can be used to assemble a nucleic acid corresponding to mannosyl transferase such as the entire mannosyl transferase gene in a host vector and, once assembled, the mannosyl transferase nucleic acid can be expressed in a suitable host. Methods for the complete chemical synthesis and assembly of genes are known in the art and described, for example, in WO9914318A1. Invention polypeptides can be isolated directly from cells that have been transformed with expression vectors as described herein. Recombinantly expressed polypeptides of the invention also can be expressed as fusion proteins with appropriate affinity tags, such as glutathione S transferase (GST) or poly His, and affinity purified. The invention polypeptide, biologically functional fragments, and functional equivalents thereof also can be produced by chemical synthesis. For example, synthetic polypeptides can be produced using Applied Biosystems, Inc. Model 430A or 431A automatic peptide synthesizer (Foster City, Calif.) employing the chemistry provided by the manufacturer.

The invention further provides an isolated nucleic acid encompassing substantially the same nucleotide sequence as shown as SEQ ID NO: 1, or a fragment thereof. The isolated nucleic acid encompassing substantially the same nucleotide sequence as shown as SEQ ID NO: 1 encodes a mannosyl transferase having substantially the same amino acid sequence as shown as SEQ ID NO: 2. Thus, the invention also provides an isolated nucleic acid encompassing substantially the same nucleotide sequence as shown as SEQ ID NO: 1, or a fragment thereof, encoding substantially the same amino acid sequence as shown as SEQ ID NO: 2, or a functional fragment thereof.

The invention also provides a nucleic acid that encompasses substantially the same nucleotide sequence as shown as SEQ ID NOS: 1, or a fragment thereof, and further contains an additional 5′ nucleic acid. Also provided is a nucleic acid that encompasses substantially the same nucleotide sequence as shown as SEQ ID NOS: 1, or a fragment thereof, and further contains an additional 3′ nucleic acid region.

The nucleic acid molecules described herein are useful for producing invention polypeptides, when such nucleic acids are incorporated into a variety of protein expression systems known to those of skill in the art. In addition, such nucleic acid molecules or fragments thereof can be labeled with a readily detectable substituent and used as hybridization probes for assaying for the presence and/or amount of a mannosyl transferase gene or mRNA transcript in a given sample as described herein. The nucleic acids described herein, and fragments thereof, also are useful as primers and templates in a PCR reaction for amplifying genes encoding invention polypeptides described herein.

Use of the term “isolated” in the present specification and claims as a modifier of DNA, RNA, polypeptides or proteins means that the DNA, RNA, polypeptides or proteins so designated have been produced in such form by the hand of man, and thus are separated from their native in vivo cellular environment. For example, an isolated nucleic acid molecule can be a molecule operatively linked to an exogenous nucleic acid sequence. An isolated nucleic acid molecule also can be a molecule removed from some or all of its normal flanking nucleic acid sequences. An isolated nucleic acid of the invention can alternatively, or additionally, be a substantially pure molecule, in that the molecule is at least 60%, 70%, 80%, 90 or 95% free from cellular components with which it is naturally associated. An isolated nucleic acid molecule can be in any form, such as in a buffered solution, a suspension, a lyophilized powder, attached to a solid support (e.g. as a component of a DNA array), or in a cell.

An isolated nucleic acid of the invention can be a polynucleotide of natural or synthetic origin, which can be single- or double-stranded, can correspond to genomic DNA, cDNA or RNA, and can represent either the sense or antisense strand or both. An isolated nucleic acid of the invention can contain one or more non-natural nucleotides, such as nucleotides having modifications to the base, the sugar, or the phosphate portion, or having one or more non-natural linkages, such as phosphothioate linkages. Such modifications can be advantageous in increasing the stability of the nucleic acid molecule, particularly when used in hybridization applications.

Furthermore, an isolated nucleic acid of the invention can be modified to contain a detectable moiety, such as a radiolabel, a fluorochrome, a ferromagnetic substance, a luminescent tag or a detectable binding agent such as biotin. A nucleic acid of the invention containing such a moiety is useful as a probe for detecting the presence or expression of a mannosyl transferase nucleic acid molecule.

As used herein, the term “substantially the same” as used in reference to a nucleic acid of the invention, refers to sequences having one or more additions, deletions or substitutions with respect to the reference sequence, so long as the nucleic acid molecule retains its ability to selectively hybridize to the reference nucleic acid molecule under moderately stringent hybridization conditions.

Moderately stringent solution hybridization conditions are conditions equivalent to hybridization in about 50% formamide, 5× Denhart's solution, 5× SSPE, 0.2% SDS at 42° C., followed by washing in 0.2× SSPE, 0.2% SDS, at 50° C. In contrast, high stringency hybridization conditions for solution hybridization can be provided, for example, by hybridization in 50% formamide, 5× Denhart's solution, 5× SSPE, 0.2% SDS at 42° C, followed by washing in 0.1× SSPE, and 0.1% SDS at 65° C. Other suitable moderate stringency and high stringency hybridization buffers and conditions are well known to those of skill in the art and are described, for example, in Sambrook et al., supra, 1989; Ausubel et al., supra, 2000.

As used herein, the term “fragment” as used in reference to a substantially pure nucleic acid molecule of the invention is intended to refer to a portion of the nucleic acid molecule having the ability to selectively hybridize with the reference nucleic acid molecule. The term “selectively hybridize” refers to an ability to bind the reference nucleic acid molecule without substantial cross-reactivity with a molecule that is not the reference nucleic acid molecule. Therefore, the term includes specific hybridization where there is little or no detectable cross-reactivity with unrelated nucleic acid molecules. The term also includes minor cross-reactivity or cross-hybridization with unrelated nucleic acid molecules, provided hybridization to the reference nucleic acid molecule is distinguishable from hybridization to the cross-reactive species. Thus, a fragment of a nucleic acid molecule of the invention can be used, for example, as a PCR primer to selectively amplify a nucleic acid molecule of the invention; as a selective primer for 5′ or 3′ RACE to determine additional 5′ or 3′ sequence of a nucleic acid molecule of the invention; as a selective probe to identify or isolate a nucleic acid molecule of the invention on a RNA or DNA blot, or genomic or cDNA library. A fragment of a mannosyl transferase nucleic acid also can encode a mannosyl transferase substrate binding domain.

A fragment having the ability to selectively hybridize can contain about 8, 9, 10, 11 or 12 nucleotides of the reference nucleic acid. A fragment also can contain a greater number of nucleotides corresponding to the reference nucleic acid, or complement thereof, including for example, about 13, 14 or 15 nucleotides as well as at least 16, 17, 18, 19 or 20 nucleotides so long as it maintains the ability to selectively hybridize to the reference nucleic acid. Additionally, a fragment can be longer, including at least about 25, 30, 40, 50, 100, 300 or 500 or more nucleotides, and can include up to the full length of the reference nucleic acid molecule minus one nucleotide. Fragments of such lengths are able to selectively hybridize with the reference nucleic acid molecule in a variety of detection formats described herein and known to those skilled in the art. A fragment of a nucleic acid of the invention can, for example, encompass a region of about 15 nucleotides having substantially the same nucleotide sequence as SEQ ID NO: 1.

The invention also provides isolated nucleic acids, each encoding a mannosyl transferase, or fragment thereof, having substantially the same nucleotide sequence as shown as SEQ ID NO: 1 and further containing a single nucleotide polymorphism. Single nucleotide polymorphisms (SNPS) are DNA sequence variations that occur when a single nucleotide in the genome sequence is changed. The nucleic acids of the invention containing single nucleotide polymorphisms shown as SEQ ID NOS: 16-117 are useful in the screening and diagnostic methods described below.

| | | | |
|---|---|---|---|
| SNP ID | Sequence | Alleles | SEQ ID NO: |

| | | | |
|---|---|---|---|
| 83889 | TAAAAGGGAACTTTTTTTGAAGAAGRATCATTTATCCAATTCACCATTCAA | AG | 16 |
| 26627 | ATTTTTTTTGTTTGTTTTTGAGATGRAGTCTCACTCTGTCGCCAGGCTGGA | AG | 17 |
| 26243 | TCCGACTCCCAGGTTCAAGTAATTCYCTTGCCTCAGCCTCCCGAGTAGCTG | TC | 18 |
| 73526 | TTATATGGGTTTGGGCCTGAATTCARATCACCTATGTGGTCCTTCAAGCCC | AG | 19 |
| 40651 | CCTGTAATCCCGGTACTTTGGGAGGYCGAGACAGGCAGATCACGAGGTCAA | TC | 20 |
| 05372 | AAAGAAGGCAGAGACTTTGTTAACTKATGATTTCTTAGCACCTAGTACCAT | TG | 21 |
| 1648389 | TTTTTAGCAATGAGCCATGTAGTTWAAAAAAAAAAAACAACTCATCTTCC | AT | 22 |
| 26894 | GAAGCCTTGAGAACACTGAATGTGTKGTCCTTTTCCTCTCACATTCGCCAC | TG | 23 |
| 564601 | ATTAGGTTTAAGAGACCCAACACAAYTGGTTCCCATTCCCACTTGTTTTTG | TC | 24 |
| 533612 | TTTCTCTTCATCACTACTAACAAACRAAACACTAAGAAGGCTTAGTATCGC | AG | 25 |
| 48033 | CGTGGACCAAAGTCTCCAGCAGAAGRGACTCATCATAAGACTGACGACTCC | AG | 26 |
| 1047400 | CAGCAGTTTCCTACAGAACACCCCCYTCCTCAATTGCCAAGGGGCCGCATC | TC | 27 |
| 595421 | TTACTTTTGATAAAGTTAATCTAAYTGTAGTTATATTTTCTGTGTGCTTT | TC | 28 |
| 523612 | TATGAGTGCAAATCATAATAGCTCCMATGTGAAAAAAAAAATCAAAAGTAT | AC | 29 |
| 524703 | GGTTTCACTCAAACCATTCCAATTRAGCCCATGCTATAGAACATTGACAG | AG | 30 |
| 61426 | GGAGCCGAGCCCCCAAAAGCTGGATRAGCAGAAAGCTCAGAAAATGTGGG | AG | 31 |
| 18138 | CACCCAGCCCTTCCTCTCACTGTGCRGCCACTGCAGGAACGGCTGGAGCTG | AG | 32 |
| 59346 | CATTTTTATTGGCATAGGTTATATRTTTGTGTGTGTGTGTGTGTGTGTGT | AG | 33 |
| 19418 | CGATACAGTAGAAGATAAAACAGACMAAAATACCAGCCTTACAGAGCTCCT | AC | 34 |
| 1053390 | TTTACTGTAAAAGTAGAAAAGCAAASACTTCAAATGATAAGACTCCAGTAT | CG | 35 |
| 15503 | AATTCAGGAAAATGTGGYTTTCATTACGGTCAAATCTCAACAT | TC | 36 |
| 599441 | TTTTTTTTTTTTTCAGGCGGAGTTTKGCCTTGTCGCCCAAGCTGGAGTGTA | TG | 37 |
| 27615 | CGATCTCAGCTCACTGCAACCTCTGMACCCTCTGCCCCCTGCCCAAGGTTC | AC | 38 |
| 580191 | TGCACCACCATGCCCGGCTAATTTTKGTATTTTTAGTAGAGACAAGGTCTT | TG | 39 |
| 21440 | GGGACTTCTTTCTGCCTACTTTAATRAACCTGGGATAGCTTCAGTTCAGAT | AG | 40 |
| 687152 | CTTTCTGCCTACTTTAATGAACCTGRGATAGCTTCAGTTCAGATTTTGCTA | AG | 41 |
| 1648390 | GGCTTCAGTTAAAGAAACATTGAAGKTCAGGTGTGGTGGCTCACGCCTGTA | TG | 42 |
| 609007 | CCTTCTGGTGGTGTCAAGAAAGGTGYGGCTCTTGAGCTGAGCCTGGAAGGA | TC | 43 |
| 663511 | TGGGATTACAGGCGTGAGCCACCGCRCCCAGCCACTTTATATCCCTTTTAA | AG | 44 |
| 680096 | ACTTAAAATGCAAATTTGGGCTAACWTTTCATGATTATATTCTTTCTTATA | AT | 45 |
| 642421 | AAGGAAAATGAAGGTAAAAAGAAGAMGGAGCATAGAGAGGGGCAAGAAAGC | AC | 46 |
| 678852 | AACTTGATAAAGATTAGCCCTAGGAKAAAAGGCTTGGTTTCAGGATTCTTC | TG | 47 |
| 623491 | TAATTGGAGAATGTTCAAAATACTTYTCTAAAGTTAATTTTTTTAGTATTC | TC | 48 |
| 681105 | GGCTGTCACTGTGAAATCTTTGATAYCCCTACCAAGAAGATCTGTGGTGGA | TC | 49 |
| 681102 | GCTGTCACTGTGAAATCTTTGATACYCCTACCAAGAAGATCTGTGGTGGAC | TC | 50 |
| 170668 | GGCACCTTTTTTTTTTTTTTTTGGAKACAGAGTCTCTCTCTGTCACCCAGG | TG | 51 |
| 1648391 | GACAGGGTTTCACCATGTTGGCCAGSCTGGTCTCGACTCCTGACCTCATG | CG | 52 |
| 229283 | TTTTTTTAAACTCTGGATATATTGTKTATTTTGTCATCCAGGCTGAGTGCA | TG | 53 |
| 170667 | GTGGTACAGTCACAGCTCACTGCAGYCTCGACCTCCCTGACTCAAACAATC | TC | 54 |
| 610437 | AAGGTGAATTTAGAAAAGGACATAARTCTTGCCCTCAGAGTCTAGTAGAAG | AG | 55 |
| 610394 | AGTCTTGCCCTCAGAGTCTAGTAGARGAAACAAACTTAAAATGTTATTAAA | AG | 56 |
| 607327 | AGATTTGTGTCCTCACTGGTTTCCTRTCAAGTTTGATATGTTGCAGGAAGA | AG | 57 |
| 645411 | ACAACCATTGGAGCTTTCTTTATTTRCCATCACCTAACTTCAGGATAGATC | AG | 58 |
| 608091 | GCCTCCCGGGTTCAAGCGATTCTCCYGCCTCAGCCTCCTGAGTAGCTGGGA | TC | 59 |
| 694177 | ACTCAAACTCATGGGCTCAAGCAATYTGCCCACCTTGGCCTCCCAAAGTGC | TC | 60 |
| 184515 | CGCCTGTAGTCCCAGCTACTCAGGARGCTGAGGCACGATAATGGCGTGAAC | AG | 61 |
| 172948 | TTTTTTTTTTTGAGACAGAGTCTTAYTCTGTTGCCCAGGCTGGAGTGCAGT | TC | 62 |
| 658186 | CTACCTATGATTCAGCAAAACTACAKCCAACGGAATGGGCTTGGCGGAGTC | TG | 63 |
| 688676 | TATGATTCAGCAAAACTACAGCCAASGGAATGGGCTTGGCGGAGTCATTGG | CG | 64 |
| 686000 | TTGTGTGTGTGTGTGTGTGTGTGTGYGCGCGCGTGTTTGGAGACAGGGTCT | TC | 65 |
| 642051 | TAGCAACACAGATTGGACTAAGACARTTGCTCTTAAGTGTGATTCTTCTGT | AG | 66 |
| 229279 | CCTTCCGCAAATACTTTTTGACTGCYTGAGATATGCCAGGTACTGTACCAG | TC | 67 |
| 1633471 | GTCAGCCATTGTGGCTGCTGGATAGYCTCAGTTATGACCAAATTTTACTTA | TC | 68 |
| 582515 | GTGAGTTTTGTTTTTTATTTTTTTAWGAGTCAGAGTCTCGCTCTGTCTCCC | AT | 69 |
| 633735 | TGTCACTATGTTGCTCAGATTGGGCHTTGATATCTTTCTAAGACCTTTACT | AC | 70 |
| 634470 | TATTAATTAGTTACCACCCACATCCSTGGTAGGCCTTGTACCGACACCAAA | CG | 71 |
| 192034 | ATCCTTTTTTTTTTTTTTTTTTTGAPACAGAGTCTTACTCTGTTGCCCAGG | AG | 72 |
| 688095 | TTTTTTGAGATAGTCTCACTCTGTCRCGACGCTGGAATGCAGAGGCACAAT | AG | 73 |
| 635148 | CATGGTGCCCAGCCTTAACAAAAAAWTTTTTTTTTGAGACGGGGTCTCGCT | AT | 74 |
| 183469 | GCACGACCTCGGCCCCAAAGTGCTAKGATTACACGCGTAAGCCACCACACC | TG | 75 |
| 694052 | TCGGCCCCAAAGTGCTAGGATTACASGCGTAAGCCACCACACCTGGCCCTT | CG | 76 |
| 172950 | ATATCCCACGATGATAAATGCAAGGMAAAAATGTATTGGGTTAAGAGGAGC | AC | 77 |
| 631318 | GCTGAGGATATAGAGAAATGCACTGWATTTGAGGTGTGAAAGAAAGGAGTC | AT | 78 |
| 622519 | ACATAGCGAACCCTGTCTCTACTAARGATACAAAAAAAATTAGCTGGGGTA | AG | 79 |
| 587755 | ACACACGCTGGAGTGCAGTGGCACASTCTTGACTCAGTGCAACCTCCACTT | CG | 80 |
| 1633469 | CCTTGTTCTTCTTCAATATTATTTYGTGGATTCTGGGTCTTTTGCCTCTC | TC | 81 |
| 643183 | TCTTTGTGTAGGGACTTGGGCTTTARTTAAGAAAGTTCAACACAGCCTGAA | AG | 82 |
| 666014 | AAGGTTTCTGCAGGGAGGTGCTCTAMTGAGTACCCGTGACCTTAAGTTGGT | AC | 83 |
| 643567 | CTGCAGGGAGGTGCTCTAATGAGTAMCCGTGACCTTAAGTTGGTAAGGAGG | AC | 84 |
| 639627 | TCACTCATCTCCAGTTAGCAAATCTYTATTGAACATTGCCAGGCAGTGTGA | TC | 85 |
| 639602 | AATCTCTATTGAACATTGCCAGGCASTGTGATCAATCATAGGTAGACAGGG | CG | 86 |
| 639591 | ATTGAACATTGCCAGGCAGTGTGATMAATCATAGGTAGACAGGGATAGCGC | AC | 87 |
| 636902 | CAATTATAGATGAAGAGTAAATACAMATAAGGAGTTAGATGTTGAACCTTT | AC | 88 |
| 614144 | GGGCAAGTTGGGGACTGTGCTGATAWCATATACGCTTATTATGTTCTTTCA | AT | 89 |
| 582465 | CTTATTATGTTCTTTCAGTGCCTACRTAGGATTCTCAGTGGCCACTGAACT | AG | 90 |
| 625017 | TGTAACAGTCATCCATACTTAGTGGYGTAAAACAACAATAGTTGATTATTG | TC | 91 |
| 614247 | AGCCCAAATGAGGGTCCGAAGAGCAKGGACGAAAACTCTCTCGAACATTGG | TG | 92 |
| 644508 | TGCCACGATTCCAGTTTGTGCCTCASTAAACTTCCTAGTCATTACACATAA | CG | 93 |
| 659786 | AGGTTGCTCCATATAGTCAAAATCAYGAATATTTATTGAACATCAAGGGTC | TC | 94 |
| 648544 | CCCCCTTGCACTCTGACATCCCATGRGCCACCACAGCCCCCCAGCTCCCCA | AG | 95 |
| 634280 | GTGGGCCAGGCATGGTGGCTCATGCSTGCAGTCCCACTATTTGGGAGGCTG | CG | 96 |
| 679465 | AAGCTTTTGATGATAAAATAATCTAKAACAGCAGCTGGAATGTTAGAAATG | TG | 97 |
| 679412 | ATGTTAGAAATGAAGAATTCAAGCCWGCTTGTTCTTTTGGTTAAPAAGAAA | AT | 98 |
| 625477 | GCAAATAAATGAATAACATGGTGTAWAGTACAAAGTAAAAGTCCTCTGCTC | AT | 99 |
| 647071 | CATTTCCCTGATTCCTGGTAAGATTSATCATTATTTCATGTTTATCAACCA | CG | 100 |
| 1054525 | CCCGGAAAGCAAAGCAAATCAGGAARAAAAGTGGAGGTTAGCAACACACCT | AG | 101 |
| 590073 | AAAGCAAATCAGGAAGAAAAGTGGARGTTAGCAACACACCTGTGGCCCCAA | AG | 102 |
| 613409 | CCCTGCAAGTCATCGCCTGTAACATKTGTAATAAAGGTCTTCTGACATGAA | TG | 103 |
| 685301 | GCTTGCCTTTGTTTTTAATATTTCTRCTCAGAAACTTCACTTGCATCTGCT | AG | 104 |
| 2007211 | GGATTACAGGTGTGAGCCACCGCACRTGGCCTCTTTTCTTTTCAAGTGCAA | AG | 105 |
| 1991303 | TGTACCTGTTTATTGACATTTTATARTGTAGGTAAATTTTTAATCAGTTTT | AG | 106 |
| 1940528 | TTGTCTTCATCAAGAAGGATGTGGAYGAAGCTTACATGAACAAAGCAGAGC | TC | 107 |
| 605923 | CCTAAGAAGACATTCAGACCCAGGTSTTATGCAGGATTACATCCGTTTATT | CG | 108 |
| 585149 | GTCTTATGCAGGATTACATCCGTTTRTTATCAAGGGCAACCTTGGTGAAAG | AG | 109 |
| 669778 | TCTCTTCAAGTGGCGGGCCCAGCCCYAGGGATGTCCAATGGCTCCCTGAGG | TC | 110 |
| 736650 | CCTCAATGATCCACACTCAGATCGGRTCATACCACGCATTCATGGCATGAG | AG | 111 |
| 647080 | TAGTGACAACACAACAGTTAACAAARGAACAAAAGTGCACCAGGTTAACCG | AG | 112 |
| 1346180 | CTTAGCATGGGAAGAGTGGGCTGTGSACTCTCATTCCCATGAGGCATAGGA | CG | 113 |
| 229284 | TGGGATCTCAACCTCTGTTTCAGGCYTTCTATTTCCTTTGAGAATGTGTAG | TC | 114 |
| 673679 | CTACTAAATTGTGAAGAGGCAAGAARATGTAACCTAAAACCAAGAAGATTT | AG | 115 |
| 654450 | GAGGGGACAGCAGATCTCAAACAACYTTCCTGGTAACAGGGGAATGCCTTC | TC | 116 |
| 634948 | GTGTTCAGGGCATGATTTTCACAACSACCTGTTGAGAAGGAAAGAATCATT | CG | 117 |

The invention further provides a isolated nucleic acid encompassing substantially the same nucleotide sequence as shown as SEQ ID NO: 3, or a fragment thereof. The invention also provides a isolated nucleic acid encompassing substantially the same nucleotide sequence as shown as SEQ ID NO: 5, or a fragment thereof. As described herein, the isolated mannosyl transferase polypeptide of the invention designated SEQ ID NO: 2 is encoded by a gene that is located on chromosome 11 and disrupted in a chromosomal translocation t(9,11) (p24;q23.1), which cosegregates with bipolar disorder. The nucleic acid encompassing substantially the same nucleotide sequence as shown as SEQ ID NO: 3, or a fragment thereof, includes a nucleic acid region from human chromosome 9 that is translocated and inserted into chromosome 11 as a result of the t(9,11) (p24;q23.1) translocation. The nucleic acid encompassing substantially the same nucleotide sequence as shown as SEQ ID NO: 5, or a fragment thereof, also includes a nucleic acid region from human chromosome 9 that is translocated and inserted into chromosome 11 as a result of the t(9,11) (p24;q23.1) translocation. The gene fusions designated SEQ ID NOS: 3 and 5, which result from the t(9,11) (p24;q23.1) translocation, have a 5′ region corresponding to chromosome 9 and a 3′ region corresponding to chromosome 11 and encode the mannosyl transferase fusion polypeptides designated SEQ ID NOS: 4 and 6, respectively. A fragment of the fusion transcripts designated as SEQ ID NOS: 3 and 5 can contain a nucleic acid region from human chromosome 9, a nucleic acid region from human chromosome 11, or can contain a 5′ nucleic acid region from human chromosome 9 and a 3′ nucleic acid region from human chromosome 11. A fragment of an isolated nucleic acid containing substantially the same nucleotide sequence as SEQ ID NOS: 3 and 5 that encompasses a 5′ nucleic acid region from human chromosome 9 and a 3′ nucleic acid region from human chromosome 11 is useful as a probe in the diagnostic methods described herein.

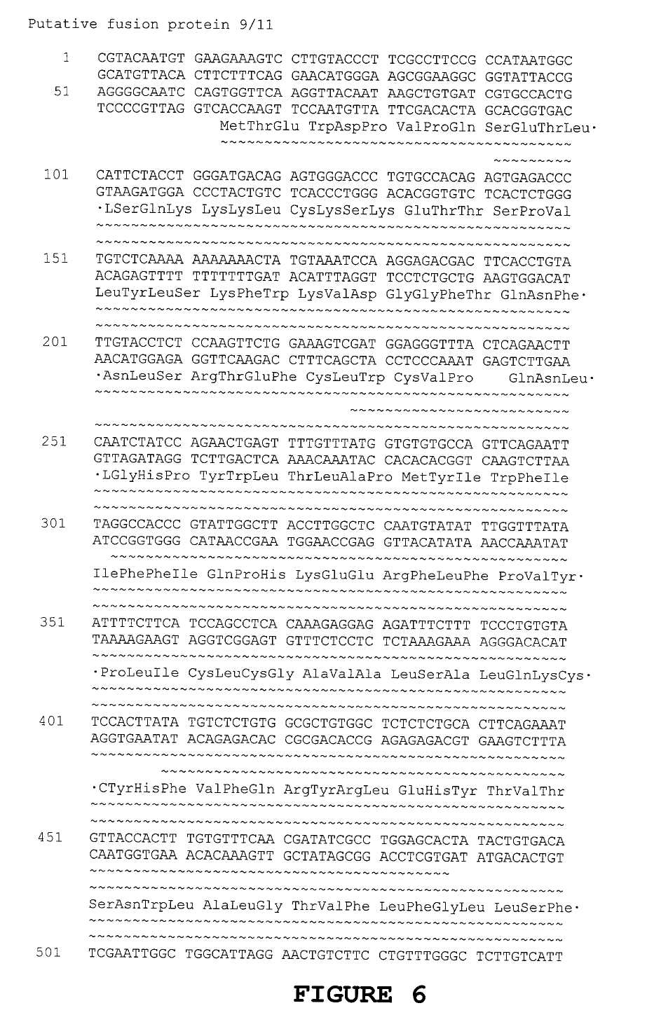

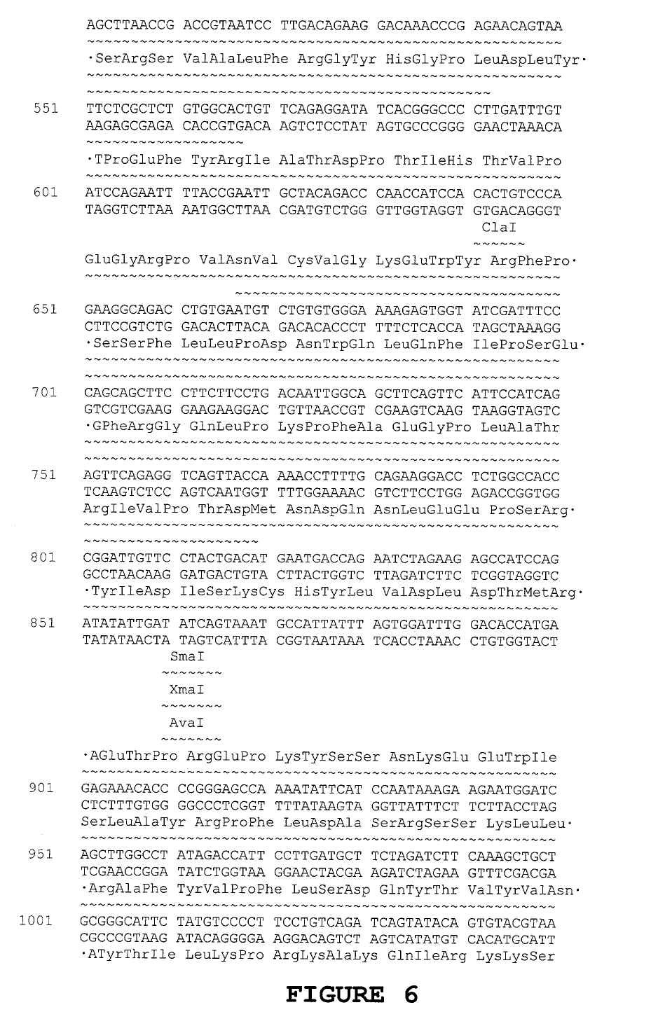

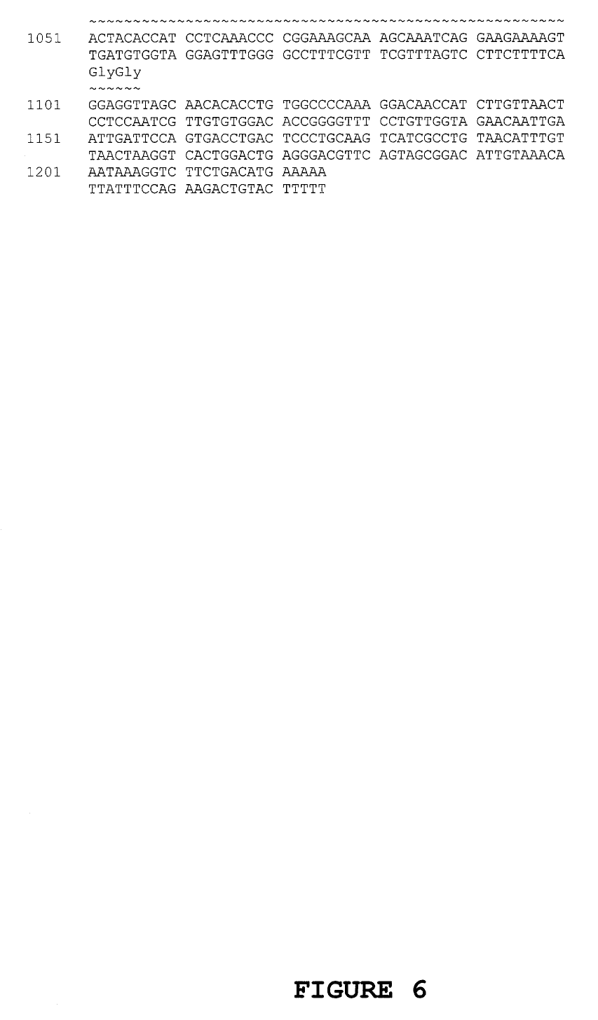

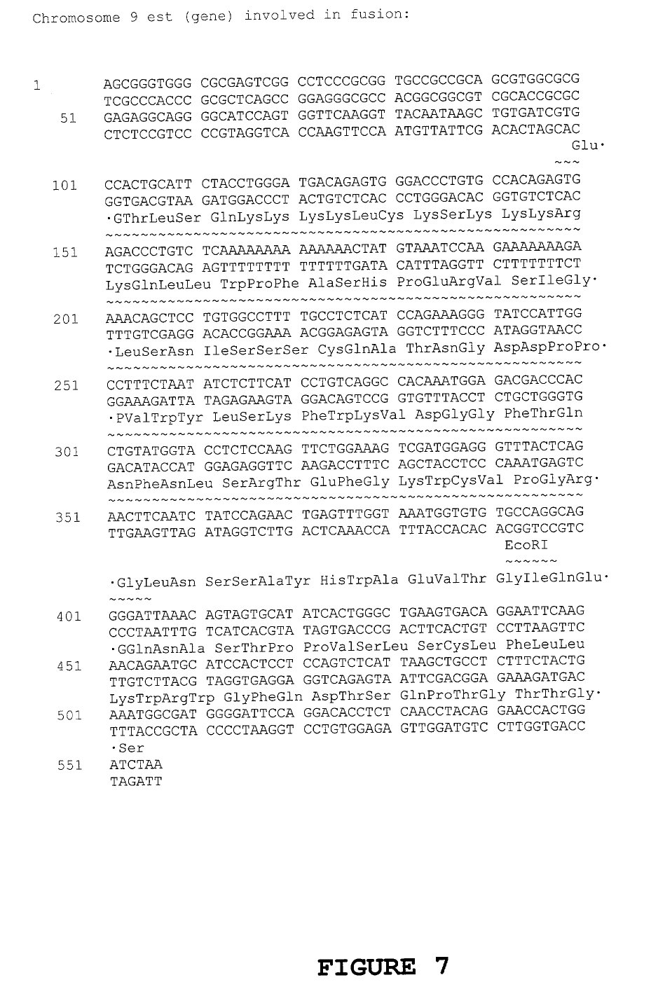

The invention provides alternative mannosyl transferase fusion polypeptides designated SEQ ID NOS: 4 and 6, and corresponding fusion nucleic acids SEQ ID NOS: 3 and 5, respectively. The precise location of the break points and resulting fusion proteins can vary, resulting in alternative fusions that vary by one or several amino acids at the fusion site and/or at both ends. Exemplary alternative forms are shown in FIGS. 3 and 6. Even if alternative fusions occur, one skilled in the art can readily select sequences from the appropriate regions of chromosome 9 or 11 to detect the translocation. In this regard, the skilled person will recognize that the precise break and fusion point can vary and can further readily confirm the exact break and fusion point by routine methods including, for example, sequencing and polymerase chain reaction.

Also provided by the invention is a nucleic acid encompassing substantially the same nucleotide sequence as shown as SEQ ID NO: 7, or a fragment thereof, which includes a nucleic acid region from human chromosome 11 that is translocated and inserted into chromosome 9 as a result of the t(9,11) (p24;q23.1) translocation. The chromosome 9 gene fusion, also referred to herein as the 11/9 gene fusion, designated SEQ ID NO: 7, which also is a result of the t(9,11) (p24;q23.1) translocation, has a 5′ region corresponding to chromosome 11 and a 3′ region corresponding to chromosome 9 and encodes the chromosome 9 fusion polypeptide designated SEQ ID NO: 8. Therefore, a fragment of the fusion transcript designated as SEQ ID NO: 7 can contain a nucleic acid region from human chromosome 9, a nucleic acid region from human chromosome 11, or can contain a 5′ nucleic acid region from human chromosome 11 and a 3′ nucleic acid region from human chromosome 9. A fragment of an isolated nucleic acid containing substantially the same nucleotide sequence as SEQ ID NO: 7 that encompasses a 5′ nucleic acid region from human chromosome 11 and a 3′ nucleic acid region from human chromosome 9 is useful as a probe in the diagnostic methods described herein. FIG. 5 shows the chromosome 9 gene fusion, or 11/9 fusion, and corresponding amino acid product.

The nucleic acids and nucleic acid fragments of the invention independently can exclude known database sequences available to the public as of the date of filing this application. In particular, a nucleic acid of the invention or fragment thereof can exclude independently the sequences available under the following Genbank Accession Numbers AK0254098 AND AL136927 as well as the following specific EST's corresponding to GenBank Accession Numbers: AL576475, BI092420, BG681522, AL516558, BF795352, BG701766, AL516557, BF308034, BF795135, BF307524, BF795137, AW273167, AW301700, AA704856, AI808501, BG674191, AA447320, AI633808, AI500656, AI050770, AW902226, BG717364, AA279575, AW964585, BG717946, AA480361, BG191573, BG620993, BI060844, BE560946, BE559584, BE397117, BE561466, AW445068, BF336986, AA886018, BF354764, BG177661, AA886008, BI113754, BE535750, BE299028, BF960307, AA321228, AI565943, AI493176, AI480277, BF960304, BF920604, AW027985, BE159877, BE829951, AI653941, AW664513, AL553488, BE736424, BE548198, AA889031, BG772576, BG720666, BG705033, BG703482, BG562917, BG333370, AI250840, AA533637, H17500, T68812, T68793, T54392. The nucleic acid sequence designated SEQ ID NOS: 3 and 5, or a fragment thereof, can further exclude independently the specific EST's corresponding to the following GenBank Accession Numbers: BF871234, BG720696, BF240568, T95528N72945, H74121, H53301, AL036499, AW876220, AW903790, AW984066, AW769064, AW419286, AI356410, AA662346, W85907, N70263, BG572712, BF842955, BF748354, BE077008, AW873440, AA584859, AA011673, R07499, Z35729, BG572043, BG536186, BF956835, BF927506, BF912327, BF893193, BF977921, BF812256, AU147929, AV726402, BE548333, BE154792, BE154749, BE154690, BE042404, BE007918, AW876425, AW876422, AW876392, AW876386, AW876382, AW876361, AW876336, AW876329, AW876325, AW876316, AW876309, AW876288, AW876267, AW876266, AW876265, AW876241, AW876238, AW876229, AW876190, AW876180, AW876176, AW876168, AW876139, AW876133, AW876124, AW817704, AW816898, AW469159, AW197229, AW152422, AI648643, AI571791, AI472699, AA730857, AA525970, AA420574, AA076760, AA001643, N35447, R77170, BG704993, BG774764, BG484054, BG476592, BG119929, BF924659, BF971055, BF513312, AW876397, AW876342, AW845746, AW473113, AW277054, AW062277, AW051339, AI370005, AI242607, AA714224.

A nucleic acid of the invention, or fragment thereof, can also include one or several nucleotide additions, deletions, or substitutions with respect to a reference sequence. A modification of a nucleic acid can include substitutions that do not change the encoded amino acid sequence due to the degeneracy of the genetic code. Such modifications can correspond to variations that are made deliberately, or which occur as mutations during nucleic acid replication.

Exemplary modifications of the recited mannosyl tranferase sequences include sequences that correspond to homologs of other species, including mammalian species such as mouse, primates, including monkey and baboon, rat, rabbit, bovine, porcine, ovine, canine, feline, or other animal species. The corresponding mannosyl transferase sequences of non-human species can be determined by methods known in the art, such as by PCR or by screening genomic, cDNA or expression libraries.

Another exemplary modification of an isolated nucleic acid of invention can correspond to splice variant forms of the mannosyl transferase nucleotide sequence. Additionally, a modification of a nucleotide sequence can include one or more non-native nucleotides, having, for example, modifications to the base, the sugar, or the phosphate portion, or having a modified phosphodiester linkage. Such modifications can be advantageous in increasing the stability of the nucleic acid molecule.

The isolated mannosyl transferase nucleic acids of the invention, which include the fusion nucleic acids of the invention, can be prepared by methods known in the art. An exemplary method for preparing an isolated mannosyl transferase nucleic acid involves computer directed synthesis of nucleic acids as known in the art and described in WO9914318A1. Alternatively, a mannosyl transferase nucleic acid can be prepared by amplification of the nucleic acid molecule using mannosyl transferase specific primers and the polymerase chain reaction (PCR). Using PCR, a mannosyl transferase nucleic acid having any desired boundaries can be amplified exponentially starting from only a few DNA or RNA molecules, such as from a single cell. PCR methods, including methods of isolating homologs of a given nucleic acid molecule in other species using degenerate primers, are well known in the art.

Alternatively, an isolated mannosyl transferase nucleic acid can be prepared by screening a library, such as a genomic library, cDNA library or expression library, with a detectable mannosyl transferase nucleic acid probe or with an antibody. Human libraries, and libraries from a large variety of mammalian species, are commercially available or can be produced from species or cells of interest. The library clones identified as containing a mannosyl transferase nucleic acid can be isolated, subcloned or sequenced by routine methods.

Furthermore, an isolated mannosyl transferase nucleic acid can be prepared by direct synthetic methods. For example, a single stranded nucleic acid molecule can be chemically synthesized in one piece, or in several pieces, by automated synthesis methods known in the art. The complementary strand can likewise be synthesized in one or more pieces, and a double-stranded molecule made by annealing the complementary strands. Direct synthesis is particularly advantageous for producing relatively short molecules, such as probes and primers, and also for producing nucleic acid molecules containing modified nucleotides or linkages.

As used herein, the term “expression level” when used in reference to a mannosyl transferase is intended to refer to the extent, amount or rate of synthesis of the nucleotide sequences shown as SEQ ID NO: 1, or the mannosyl transferase polypeptide shown as SEQ ID NO: 2. The amount or rate of synthesis can be determined by measuring the accumulation or synthesis of mannosyl transferase RNA, mannosyl transferase polypeptide or by measuring mannosyl transferase polypeptide activity. As used herein, the term “acitivity level” when used in reference to a mannosyl transferase polypeptide is intended to refer to the catalytic activity with regard to the glycosylation reaction catalyzed by the mannosyl transferase. Activity can be measured, for example, by the rate of conversion or turnover of the monosaccharide substrate, by the rate of formation of the product oligosaccharide or by the formation or accumulation of an intermediate of the glycosylation reaction.

The invention further provides a method of diagnosing or predicting the susceptibility of a bipolar disorder, by contacting a sample obtained from an individual with a probe selective for an altered mannosyl transferase sequence, and detecting binding of the probe to an analyte in the sample, wherein specific binding of the probe indicates the presence of an altered mannosyl transferase and occurrence or susceptibility of a bipolar disorder.

As used herein, the term “bipolar disorder” refers to a condition, also known as manic depressive disease, that is characterized by recurrent mood episodes of excessive highs (mania) to profound hopelessness (depression), usually with periods of normal mood in between. The type, severity and duration of mood episodes associated with bipolar disorder can vary. It is understood that some individuals can have a predominance of either mania or depression, whereas other sufferers can experience equal numbers of both. The mood episodes can last for a few days to as long as several months. Bipolar disorder encompasses bipolar I disorder, bipolar II disorder, hypomania, mixed episodes, cycylothymia, and dysthymia. Recurrent unipolar depression with bipolar family history, a condition known as bipolar disorder III and Attention Deficit Hyperactivity Disorder (ADHD), also are encompassed by bipolar disorder. The methods herein are applicable for the diagnosis or treatment of any or all stages of bipolar disorder.

The methods of the invention are also applicable to psychiatric disorders other than bipolar disorder including, for example, unipolar disorder. Unipolar disorder (major depressive disorder) is the leading cause of disability in the United States and worldwide with 19 million adults afflicted in the United States alone. Other affective disorders, including secondary affective states, known to those skilled in the art exist as well and are similarly applicable for diagnosis or treatment using the methods of the invention. Various psychiatric disorders can be found described in, for example, Diagnostic and Statistical Manual of Mental Disorders (DSM IV-R), (Revised, Fourth Edition, American Psychiatric Association, 1992) and Diagnostic and Statistical Manual of Mental Disorders, Text Revision (Text Revision, American Psychiatric Association, 2000). Therefore, the methods of the invention are applicable to bipolar disorder as well as other psychiatric disorders.

As used herein, the term “sample” is intended to mean any biological fluid, cell, tissue, organ or portion thereof, that includes or potentially includes nucleic acids and polypeptides of the invention. The term includes samples present in an individual as well as samples obtained or derived from the individual. For example, a sample can be a histologic section of a specimen obtained by biopsy, or cells that are placed in or adapted to tissue culture. A sample further can be a subcellular fraction or extract, or a crude or substantially pure nucleic acid or protein preparation. Since the mannosyl transferase polypeptide is embedded in the membrane of the endoplasmatic reticulum (ER), samples for detection of the polypeptide will be in a format that makes accessible for contacting the membranes of the ER such as, for example, as cell fractionate or lysate containing membranes. A sample can be prepared by methods known in the art suitable for the particular format of the detection method.

As used herein, the term “probe” refers to a portion or fragment of a nucleic acid or polypeptide that has the ability to selectively bind a target molecule.

A probe selective for an altered mannosyl transferase can have substantially the same nucleotide sequence as shown as SEQ ID NO: 1, SEQ ID NO: 3, SEQ ID NO: 5 and SEQ ID NO: 7, or fragment of SEQ ID NO: 1, SEQ ID NO: 3, SEQ ID NO: 5 and SEQ ID NO: 7. A fragment of the nucleic acid probe having substantially the same nucleotide sequence as shown as SEQ ID NO: 3 can have a 5′ nucleic acid region from human chromosome 9 and a 3′ nucleic acid region from human chromosome 11. Similarly, fragment of the nucleic acid probe having substantially the same nucleotide sequence as shown as SEQ ID NO: 5 also can have a 5′ nucleic acid region from human chromosome 9 and a 3′ nucleic acid region from human chromosome 11. A fragment of the nucleic acid probe having substantially the same nucleotide sequence as shown as SEQ ID NO: 7 can have a 5′ nucleic acid region from human chromosome 11 and a ₃′ nucleic acid region from human chromosome 9. A fragment of the nucleic acid probe having substantially the sequence of SEQ ID NOS: 1, 3, 5 or 7 also can be an oligonucleotide of about 15-18 nucleotides in length. The nucleic acid probes of the invention can be produced recombinantly or synthesized using methods well known in the art. Additionally, mannosyl transferase hybridization probes can be labeled with a variety of detectable labels including, for example, radioisotopes, fluorescent tags, reporter enzymes, biotin and other ligands. Such detectable labels can additionally be coupled with, for example, colorimetric indicator substrate for spectrophotometric detection. Methods for labeling and detecting such probes are well known in the art and can be found described in, for example, Sambrook et al., supra, 1989; Ausubel et al., supra, 2000.

The expression level of a mannosyl transferase in normal cells and cells obtained from an individual suspected of having bipolar disorder can be measured using a probe selective for an altered mannosyl transferase nucleic acid. For example, a probe selective for an altered mannosyl transferase sequence can be a complementary nucleic acid, such as a hybridization probe having substantially the nucleotide sequence as shown as SEQ ID NOS: 1, 3, 5, 7, or fragment thereof, or a non-catalytic ribozyme that selectively hybridizes to a mannosyl tranferase nucleic acid. A hybridization probe or ribozyme can be labeled with a detectable moiety, such as a radioisotope, fluorochrome, chemiluminescent marker, biotin, or other detectable moiety known in the art that is detectable by an analytical method. A PCR or RT-PCR primer also can be used to detect a mannosyl transferase nucleic acid. For example, a PCR or RT-PCR primer can be used to selectively amplify all or a desired portion of the nucleic acid molecule, which can then be detected by methods known in the art. Further, a binding agent such as a peptide, nucleic acid analog, or small organic molecule can be used to detect a mannosyl transferase nucleic acid. Selective hybridization refers to the hybridization of a probe or binding agent to a mannosyl transferase nucleic acid.

As used herein, the term “binding agent” is intended to mean a compound, a macromolecule, including polypeptide, DNA, RNA and carbohydrate that selectively binds a polypeptide of the invention, or a fragment thereof. For example, a binding agent can be a polypeptide that selectively binds with high affinity or avidity to a mannosyl transferase having substantially the same amino acid sequence as shown as in SEQ ID NOS: 2, without substantial cross-reactivity with other unrelated polypeptides. The affinity of a binding agent that selectively binds a mannosyl transferase having substantially the same amino acid sequence as shown as in SEQ ID NOS: 2, will generally be greater than about 10⁻⁵ M and more preferably greater than about 10⁻⁶ M. High affinity binding interactions will generally be greater than about 10⁻⁸ M to 10⁻⁹ M. A binding agent can further be a polypeptide that selectively binds with high affinity or avidity to a fusion polypeptide having substantially the same amino acid sequence as shown as in SEQ ID NOS: 4, 6, or 8, without substantial cross-reactivity with other unrelated polypeptides. Specific examples of such selective binding agents include a polyclonal or monoclonal antibody specific for a mannosyl transferase having substantially the same amino acid sequence as shown as in SEQ ID NO: 2, or a functional fragment thereof, or a polypeptide, nucleic acid, nucleic acid analog, or small organic molecule, identified, for example, by affinity screening of a library with a mannose transferase polypeptide or fragment. Further specific examples of such selective binding agents include a polyclonal or monoclonal antibody specific for a fusion polypeptide of the invention having substantially the same amino acid sequence as shown as in SEQ ID NOS: 4, 6, or 8, or a functional fragment thereof, For certain applications, a binding agent can be utilized that preferentially recognizes a particular conformational or post-translationally modified state of a polypeptide of the invention. The binding agent can be labeled with a detectable moiety, if desired, or rendered detectable by specific binding to a detectable secondary binding agent.

A probe selective for an altered mannosyl transferase sequence can be a binding agent having having specific binding activity to a polypeptide encoded by substantially the same nucleotide sequence as shown as SEQ ID NOS: 3 and 5, or a fragment thereof. The polypeptide encoded by the nucleotide sequence shown as SEQ ID NOS: 3 and 5 is a fusion protein encoded by a fusion transcript that encompasses a 5′ nucleic acid region from human chromosome 9 and a 3′ nucleic acid region from human chromosome 11. A probe selective for an altered mannosyl transferase sequence also can be a binding agent having specific binding activity to a polypeptide encoded by substantially the same nucleotide sequence as shown as SEQ ID NO: 7, or a fragment thereof. The polypeptide encoded by the nucleotide sequence shown as SEQ ID NO: 7 and shown in FIG. 5 is a fusion protein encoded by a fusion transcript that encompasses a 5′ nucleic acid region from human chromosome 11 and a 3′ nucleic acid region from human chromosome 9. The invention fusion proteins, which are designated as SEQ ID NOS: 4, 6 and 8, respectively, result from the chromosomal translocation t(9,11) (p24;q23.1) that cosegregates with bipolar disorder. A probe selective for an altered mannosyl transferase sequence that is a binding agent having specific binding activity to a polypeptide encoded by substantially the same nucleotide sequence as shown as SEQ ID NOS: 3, 5 or 7, or a fragment thereof, can be an antibody that recognizes an epitope present on either of the fusion polypeptides designated SEQ ID NOS: 4, 6 and 8, respectively. Preferably, the antibody is provided purified and as a homogeneous preparation.

An antibody of the invention, or antigen binding fragment of such an antibody, is characterized by having specific binding activity for a target molecule or a fragment thereof of at least about 1×10⁵ M⁻¹. Thus, Fab, F(ab′)₂, Fd and Fv fragments of an invention antibody, which retain specific binding activity for a target molecule, are included within the definition of an antibody. Specific binding activity of an antibody to a target molecule can be readily determined by one skilled in the art, for example, by comparing the binding activity of an antibody to a target molecule versus a control molecule that is not the target molecule. Methods of preparing polyclonal or monoclonal antibodies are well known to those skilled in the art (see, for example, Harlow and Lane, Using Antibodies: A Laboratory Manual, Cold Spring Harbor Laboratory Press, New York (1999)).

In addition, an antibody of the invention can be naturally occurring as well as non-naturally occurring and can be a single chain antibody, chimeric, bifunctional or humanized antibody, as well as an antigen-binding fragment thereof. Such a non-naturally occurring antibody can be constructed using solid phase peptide synthesis, can be produced recombinantly or can be obtained, for example, by screening combinatorial libraries consisting of variable heavy chains and variable light chains as described by Huse et al. (Science 246:1275-1281 (1989)). These and other methods of making, for example, a chimeric, humanized, CDR-grafted, single chain, or bifunctional antibody are well known to those skilled in the art (Winter and Harris, Immunol. Today 14:243-246 (1993); Ward et al., Nature 341:544-546 (1989) ; Harlow and Lane, supra, 1999); Hilyard et al., Protein Engineering: A practical approach (IRL Press 1992); Borrabeck, Antibody Engineering, 2d ed. (Oxford University Press 1995)). An antibody that recognizes an epitope present on a mannosyl transferase fusion polypeptide (SEQ ID NOS: 4 and 6) or a chromosome 9 fusion polypeptide (SEQ ID NO: 8) shown in FIG. 5) encoded by the by substantially the same nucleotide sequence as shown as SEQ ID NOS: 3, 5, and 7, respectively, or a fragment thereof, is useful as a probe for an altered mannosyl transferase sequence.

Therefore, the invention provides a method of diagnosing or predicting the susceptibility of a bipolar disorder, by contacting a sample obtained from an individual with a probe selective for an altered mannosyl transferase sequence, and detecting binding of the probe to an analyte in the sample, wherein specific binding of the probe indicates the presence of an altered mannosyl transferase and occurrence or susceptibility of a bipolar disorder.

The invention also provides a method of diagnosing or predicting the susceptibility of a bipolar disorder. The method consists of measuring an expression or activity level of mannosyl transferase in a sample obtained from an individual, and comparing the measured expression or activity level of mannosyl transferase to mannosyl transferase expression or activity levels from a reference sample, wherein a reduction of the measured expression or activity levels compared to the reference sample indicates the occurrence or susceptibility of a bipolar disorder.

As used herein, the term “expression level” is intended to refer to the extent, amount or rate of synthesis of the nucleotide sequence shown as SEQ ID NO: 1, or the mannosyl transferase polypeptide shown as SEQ ID NO: 2. The amount or rate of synthesis can be determined by measuring the accumulation or synthesis of mannosyl transferase RNA, polypeptide or by measuring mannosyl transferase polypeptide activity.

The invention provides a method of diagnosing or predicting the susceptibility of a bipolar disorder based on the finding of a positive correlation between disruption of the mannosyl transferase gene and bipolar disorder. As disclosed herein, the human mannosyl transferase gene is disrupted in a chromosomal translocation t(9,11) (p24;q23.1) that cosegregates with bipolar disorder. Therefore, changes in mannosyl transferase expression or activity can be correlated with bipolar disorder and, further, can be used to predict the susceptibility to bipolar disorder. One consequence of progression into bipolar disorder is a decrease in the expression or activity of mannosyl transferase. A decrease in mannosyl transferase expression in individuals suffering from a bipolar disorder can be measured by comparing the amount of mannosyl transferase in a sample obtained from an individual to that found, for example, in tissue samples obtained from non-afflicted individuals. A statistically significant decrease in the expression level of a mannosyl transferase is indicative of a bipolar disorder.

As a diagnostic indicator, mannsosyl transferase expression can be used qualitatively to positively identify a bipolar disorder as described above. Alternatively, mannsosyl transferase expression also can be used quantitatively to determine the degree or susceptibility of a bipolar disorder. In this regard, the lower the level of mannosyl transferase expression, the more severe the bipolar disorder. For example, decreases in expression levels of 25% or more compared to a normal sample is indicative of severe form of bipolar disorder. A decrease in expression along the spectrum by 10%, 20%, 30%, 40% or 50% would be expected to represent mild to severe bipolar symptoms. Mannosyl transferase expression also can be used qualitatively to distinguish between a genetically based bipolar disorder and an environmental or non-genetic mood episode or disorder. Correlative decreases can be determined by comparison of mannosyl transferase expression from the individual having, or suspected of having a bipolar disorder to expression levels from samples of an individual determined to suffer from a bipolar disorder. Alternatively, correlative increases also can be determined by comparison of mannosyl transferase expression from the test individual to expression levels of other known genetic markers of a bipolar disorder

The use of mannosyl transferase expression levels allows for early diagnosis as a predictive indicator when either no definitive pathological symptoms or are apparent or in an asymptomatic individual. The methods are applicable to any individual with a familial history of affective disorder. The diagnostic methods of the invention also are applicable to individuals predicted to be at risk for bipolar disorder by reliable prognostic indicators prior to onset of overt clinical symptoms. All that is necessary is determination of the mannosyl transferase expression levels to determine whether there is an decrease in these mannosyl transferase levels in the individual suspected of having a or being susceptible to a bipolar disorder compared to normal individuals. Those skilled in the art will know by using routine examinations and practices in the fields of psychiatry and psychology those individuals who are applicable candidates for diagnosis by the methods of the invention.

For example, individuals suspected of having a bipolar disorder can be identified by exhibiting mood swings from excessive highs (mania) to profound hopelessness (depression), usually with periods of normal mood in between. Some individuals may exhibit mixed symptoms of both mania and depression at the same time, while others may have more moderate symptoms of mania (hypomania). The presenting signs of mania are well known to those skilled in the art and include, for example, increased energy and activity, restlessness, racing thoughts and rapid speech, excessive euphoria, extreme irritability and distractability, decreased sleep requirement, uncharacteristically poor judgement, overspending and risk-behavior. The presenting signs of depression also are well known to those skilled in the art and include, for example, feeling sad or anxious for large portions of the every day, tearfulness or frequent weeping or crying, loss of interest in formerly enjoyed activities, weight changes not due to dieting or medication side effects, significant changes in sleep patterns including both hypersomnia and insomnia, psychomotor agitation or retardation, lack of energy, feelings of worthlessness or hopeless, indecisiveness, thoughts of suicide or death, hallucinations or delusions. Prognostic methods of this invention are applicable to individuals after diagnosis of a bipolar disorder, for example, to confirm the diagnosis or determine the basis of the bipolar disorder.

The diagnostic methods of the invention are applicable for use with a variety of different types of samples isolated or obtained from an individual having, or suspected of having a bipolar disorder. For example, samples applicable for use in one or more diagnostic formats of the invention, include tissue and cell samples. A tissue or cell sample can be obtained, for example, by biopsy or surgery. As described below, and depending on the format of the method, the tissue can be used whole or subjected to various methods known in the art to disassociate the sample into smaller pieces, cell aggregrates or individual cells. Additionally, when combined with amplification methods such as polymerase chain reaction (PCR), a single cell sample is sufficient for use in diagnostic assays of the invention which employ hybridization detection methods. Similarly, when measuring mannosyl transferase polypeptide or activity levels, amplification of the signal with enzymatic coupling or photometric enhancement can be employed using only a few or a small number of cells.

Whole tissue obtained from a biopsy or surgery is one example of a cell sample suitable for the methods of the invention. Whole tissue cell samples can be assayed employing any of the formats described below. For example, the tissue sample can be mounted and hybridized in situ with mannosyl transferase nucleic acid probes. Similar histological formats employing protein detection methods and in situ activity assays also can be used to detect mannosyl transferase polypeptides in whole tissue cell samples. Protein detection methods include, for example, staining with mannosyl transferase specific antibodies and activity assays which result in the deposition of a mannosyl transferase catalytic product at the site of glycosylation activity in the sample. Such histological methods as well as others well known to those skilled in the art are applicable for use in the diagnostic methods of the invention using whole tissue as the source of a cell sample. Methods for preparing and mounting the samples are similarly well known in the art.

As described above, N-linked glycosylation in eukaryotes is initiated in the endoplasmatic reticulum. N-linked oligosaccharides are attached to their polypeptide chain by an -N-glycosidic bond to an Asparagine (Asn) residue in the sequence Asn-X-Ser or Asn-X-Thr, where X is any amino acid residue except Proline or Aspartic Acid. N-linked oligosaccharides result from synthesis of a lipid-linked oligosaccharide precursor and subsequent transfer of this precursor to the NH₂ group of an Asn residue on a growing polypeptide, which is followed by removal of some of the precursor's sugar units and addition of sugar residues to the remaining core oligosaccharide. Dolichol is the lipid component in this process that anchors the growing oligosaccharide to the ER membrane. Dolichol is a highly hydrophobic lipid that resides within the ER membrane, with its active site facing the lumen. The oligosaccharide is added by sugar residues individually; it is linked to dolichol by a pyrophosphate group, and is subsequently transferred as a unit to a target protein by a membrane bound glycosyl transferase enzyme. In particular, at first two GlcNAc and five Man residues, derived from UDP-GlcNAc and GDP-Man, respectively, are added. Subsequently, seven sugars, specifically four Man and three Glc residues, provided by the lipid intermediates dolichol (Dol)-P-Man and Dol-P-Glc, respectively, are added. Each transfer reaction is mediated by an individual gycosyltransferase in the ER (Kornfeld and Kornfeld, supra, 1985; Tanner and Lehle, supra, 1987; Herscovics and Orlean, supra, 1993; Kukuruzinska et al., supra, 1987). Glycoprotein processing is subsequently completed in the Golgi Apparatus.

The mannosyl transferase polypeptide of the invention, which contains nine transmebrane domains, is embedded in the ER membrane. Consequently, fractions containing cell membranes that encompass organelle membranes, organelle lysates and micelle preparations obtained from organelle membranes of individual cells and cell aggregates from an individual having, or suspected of having a bipolar disorder also are examples of cell samples that can be analyzed for increased expression of mannosyl transferase RNA, polypeptide or activity. The cells can be grown in culture and analyzed in situ using procedures such as those described above. Generally, however, cell samples expressing cell surface markers such as mannosyl transferase can rapidly be tested using fluorescent or magnetic activated cell sorting (FACS or MACS) with labeled binding agents specific for mannosyl transferase or using binding agents selective for endoplasmatic reticulum markers, for example, and then determining the expression level of mannosyl transferase within this population. The expression level can be determined by, for example, binding agents specific for mannosyl transferase or by hybridization to a mannosyl transferase specific probe. Other methods for measuring the expression level of mannosyl transferase in cell samples containing organelle membrane fractions are known in the art and similarly are applicable in any of the diagnostic formats herein.

The tissue or cell sample obtained from an individual can be analyzed for mannosyl transferase expression by lysing the cell and measuring the expression levels of mannosyl transferase in the lysate, which will contain the cell organelles, a fractionated portion of such a lysate or a purified component thereof using any of diagnostic formats described below. For example, if a hybridization format is used, mannosyl transferase RNA can be amplified directly from the lysate using PCR, or other amplification procedures well known in the art such as RT-PCR, 5′ or 3′ RACE to directly measure the expression levels of mannosyl transferase. RNA also can be isolated and probed directly such as by solution hybridization or indirectly by hybridization to immobilized RNA. Similarly, when determining the expression level of mannosyl transferase using polypeptide detection or glycosylation activity formats, lysates can be assayed directly, or can be further fractionated to enrich for mannosyl transferase and its corresponding activity. Numerous other methods applicable for use with cell samples are well known to those skilled in the art and can accordingly be used in the methods of the invention.

The cell tissue or cell sample can be obtained directly from the individual or, alternatively, it can be obtained from other sources for testing. Similarly, the cell sample can be tested when it is freshly isolated or it can be tested following short or prolonged periods of cryopreservation without substantial loss in accuracy or sensitivity. If the sample is to be tested following an indeterminate period of time, it can be obtained and then cryopreserved, or stored at 4° C. for short periods of time, for example. An advantage of the diagnostic methods of the invention is that they do not require histological analysis of the sample. As such, the sample can be initially disaggregated, lysed, fractionated or purified and the active component stored for later diagnosis.

A bipolar disorder can be diagnosed, predicted or prognosed by measuring the expression level of mannsosyl transferase in a cell sample, including a cell fractionate or lysate, obtained from the individual provided the sample contains organelle membranes. As described above, expression levels can be measured by a variety methods known in the art. For example, the expression level of mannosyl transferase can be determined by measuring the expression level of a mannosyl transferase RNA or polypeptide in a sample from the individual. Alternatively, the expression level of mannosyl transferase can be determined by measuring the amount of mannosyl transferase activity in the sample, the amount of activity being indicative of mannosyl transferase expression levels. A decrease in the expression or activity level of a mannosyl transferase can be detemined by detecting the presence of hypoglycosylated secretory proteins. In particular, hypoglycosylated secretory proteins characterized by a reduction in N-linked glycosylation are indicative of a decrease in mannosyl transferase expression or activity levels.

Given the teachings and guidance provided herein, the choice of measuring RNA, polypeptide or activity will be that of the user. Considerations such as the sample type, availability and amount will also influence selection of a particular diagnostic format. For example, if the sample is a cell sample and there is only a small amount available, then diagnostic formats which measure the amount of mannosyl transferase RNA by, for example, PCR amplification, or which measure a mannosyl transferase polypeptide by, for example, antibody detection in a cell fractionate can be appropriate choices for determining the expression level of a mannosyl transferase. Additionally, mannosyl transferase nucleic acids also can be analyzed by, for example, PCR or RT-PCR. Those skilled in the art will know, or can determine which format is amenable for a particular application and which methods or modifications known within the art are compatible with a particular type of format.

Hybridization methods are applicable for measuring the amount of mannosyl transferase RNA as an indicator of mannosyl transferase expression levels. There are numerous methods well known in the art for detecting nucleic acids by specific or selective hybridization with a complementary probe. Such methods include both solution hybridization procedures and solid-phase hybridization procedures where the probe or sample is immobilized to a solid support. Descriptions for such methods can be found in, for example, Sambrook et al., supra, and in Ausubel et al., supra, 2000. Specific examples of such methods include PCR and other amplification methods such as RT-PCR, 5′ or 3′ RACE, RNase protection, RNA blot, dot blot or other membrane-based technologies, dip stick, pin, ELISA or two-dimensional arrays immobilized onto chips as a solid support. These methods can be performed using either qualitative or quantitative measurements, all of which are well known to those skilled in the art.

PCR or RT-PCR can be used with isolated RNA or crude cell lysate preparations. As described previously, PCR is advantageous when there is little starting material. A further description of PCR methods can be found in, for example, Dieffenbach, C. W. , and Dveksler, G. S. , PCR Primer: A Laboratory Manual, Cold Spring Harbor Press, Plainview, N.Y. (1995). Multisample formats such as an ELISA or two-dimensional array offer the advantage of analyzing numerous, different samples in a single assay.

Nucleic acid probes useful for measuring the expression level of mannosyl transferase by hybridization include, for example, the mannosyl transferase nucleic acid probes described herein. More specifically, such probes include, for example, nucleic acids corresponding to the entire mannosyl transferase transcript (SEQ ID NO: 1) and fragments thereof such as the substrate binding domain. Probes useful to detect the expression level of a mannosyl transferase similarly can contain substantially the same nucleotide sequence as shown as SEQ ID NO: 1, or fragments thereof. Smaller fragments of the nucleic acid probes of the invention also can be used including, for example, short oligonucleotides corresponding to mannosyl transferase nucleotide sequences and which are capable of specifically or selectively hybridizing to mannosyl transferase RNA.

Briefly, for detection by hybridization, mannosyl transferase nucleic acid probes having detectable labels are added to a cell sample obtained from the individual having, or suspected of having a bipolar disorder under conditions which allow annealing of the probe to mannosyl transferase RNA. As described herein, selective hybridization conditions are well known in the art for both solution and solid phase hybridization procedures. Moreover, optimization of hybridization conditions can be performed, if desired, by hybridization of an aliquot of the sample at different temperatures, durations and in different buffer conditions. Such procedures are routine and well known to those skilled. Following annealing, the sample is washed and the signal is measured and compared with a suitable control or standard value. The magnitude of the hybridization signal is directly proportional to the expression levels of mannosyl transferase.

A suitable control for comparison can be, for example, the expression level of mannosyl transferase from a cell sample obtained from a normal individual. The control sample for comparison can be measured simultaneously with one or more test samples or, alternatively, expression levels can be established for a particular type of sample and standardized to internal or external parameters such as protein or nucleic acid content, cell number or mass of tissue. Such standardized control samples can then be directly compared with results obtained from the test sample. A decrease of about 20% or more of mannosyl transferase expression levels indicates the presence of a bipolar disorder or susceptibility thereto in the tested individual.

The diagnostic procedures described above and below can additionally be used in conjunction with other diagnostic methods, including other known genetic markers for simultaneous or independent corroboration of a sample. Those skilled in the art will know which markers are applicable for use in conjunction with mannosyl transferase to delineate more specific diagnostic information such as that described above.

Also provided by the present invention are diagnostic systems, generally in kit form, comprising at least one probe selective for an altered mannosyl transferase sequence in a suitable packaging material. A suitable diagnostic system includes at least one invention mannosyl transferase nucleic acid, mannosyl transferase polypeptide, and/or anti-mannosyl transferase antibody, generally two or more invention nucleic acids, polypeptides and/or antibodies, as a separately packaged chemical reagent(s) in an amount sufficient for at least one assay. Instructions for use of the packaged reagent are also typically included. Those of skill in the art can readily incorporate invention nucleic probes and/or primers into kit form in combination with appropriate buffers and solutions for the practice of the invention methods as described herein. For example, the kit can contain a nucleic acid probe of the invention having substantially the same nucleotide sequence as shown as SEQ ID NOS: 1, 3, 5 or 7, or a binding agent having selective binding activity to a polypeptide encoded by substantially the same nucleotide sequence as shown as SEQ ID NOS: 3, 5, or 7, or a fragment thereof. A binding agent having selective binding activity to a polypeptide encoded by substantially the same nucleotide sequence as shown as SEQ ID NOS: 3, 5, or 7, or a fragment thereof, can be, for example, an antibody. In addition, a kit can contain a nucleic acid probe of the invention having substantially the same nucleotide sequence as shown as SEQ ID NOS: 16 to 117, or a fragment thereof, or a binding agent having selective binding activity to a binding agent selective for one or more SEQ ID NOS: 16 to 117, or a fragment thereof. Invention diagnostic systems are useful for assaying for the presence or absence of an altered mannosyl transferase in a sample. In addition, a diagnostic system of the invention can contain materials useful for the isolation of cells and preparation of a cell lysate or a cell fractionate from a sample.

The contents of the kit of the invention are contained in packaging material, generally to provide a sterile, contaminant-free environment. In addition, the packaging material contains instructions indicating how the materials within the kit can be employed to isolate a particular cell fraction or prepare a lysate, if appropriate. The instructions for use typically include a tangible expression describing the reagent concentration or at least one assay method parameter, such as the relative amounts of reagent and sample to be admixed, maintenance time periods for reagent/sample admixtures, temperature, buffer conditions, and the like.

Therefore, the invention provides a method of diagnosing or predicting the susceptibility of a prostate neoplastic condition in an individual suspected of having a neoplastic condition of the prostate where mannosyl transferase expression level is determined by measuring the amount of mannosyl transferase nucleic acid. The amount of mannosyl transferase nucleic acid can be determined by hybridization with a nucleic acid probe having substantially the nucleotide sequence of SEQ ID NO: 1, or fragment thereof, and wherein the fragment consists of an oligonucleotide of about 15-18 nucleotides in length.

The invention additionally provides a method of diagnosing or predicting the susceptibility of a bipolar disorder by measuring the expression of mannosyl transferase polypeptide. The method consists of contacting a cell, a cell lysate, or fractionated sample thereof, from the individual with a binding agent selective for mannosyl transferase, and determining the amount of selective binding of the agent. The fractionated sample can be lipid membranes and the binding agent can be an antibody or substrate analog inhibitor.

As used herein, the term “substrate analog inhibitor” when used in reference to a mannosyl transferase is intended to mean any agent which, in the presence of mannosyl transferase, has about the same binding activity with mannosyl transferase as the naturally occurring monosaccharide substrate, but does not get converted into a measurable product. Such inhibitors can include competitive, non-competitive and covalent inhibitors. A substrate analog inhibitor is useful as a binding agent to detect the expression of a mannosyl transferase polypeptide or functional fragment thereof.

Essentially all modes of affinity binding assays are applicable for use in determining the amount of mannosyl transferase polypeptide in a sample. Such methods are rapid, efficient and sensitive. Moreover, affinity binding methods are simple and can be adjusted to be perform under a variety of clinical settings and conditions to suit a variety of particular needs. Affinity binding assays which are known and can be used in the methods of the invention include both soluble and solid phase formats. A specific example of a soluble phase affinity binding assay is immunoprecipitation using a mannosyl transferase selective antibody or other binding agent. Solid phase formats are advantageous for the methods of the invention since they are rapid and can be performed more easily on multiple different samples simultaneously without losing sensitivity or accuracy. Moreover, solid phase affinity binding assays further are amenable to high throughput screening and automation.

Specific examples of solid phase affinity binding assays include immunoaffinity binding assays such as an ELISA and radioimmune assay (RIA). Other solid phase affinity binding assays are known to those skilled in the art and are applicable to the methods of the invention. Although affinity binding assays are generally formatted for use with an antibody binding molecules that is selective for the analyte or ligand of interest, essentially any binding agent can be alternatively substituted for the selectively binding antibody. Such binding agents include, for example, macromolecules such as polypeptides, peptides, nucleic acids, lipids and sugars as well as small molecule compounds. Other binding agents selective for mannosyl transferase include, for example, mannosyl transferase substrate analog inhibitors. Methods are known in the art for identifying such molecules which bind selectively to a particular analyte or ligand and include, for example, surface display libraries and combinatorial libraries. Thus, for a molecule other than an antibody to be used in an affinity binding assay, all that is necessary is for the binding agent to exhibit selective binding activity for mannosyl transferase.

Various modes of affinity binding formats are similarly known which can be used in the diagnostic methods of the invention. For the purpose of illustration, particular embodiments of such affinity binding assays will be described further in reference to immunoaffinity binding assays. The various modes of affinity binding assays, such as immunoaffinity binding assays, include for example, solid phase ELISA and RIA as well as modifications thereof. Such modifications thereof include, for example, capture assays and sandwich assays as well as the use of either mode in combination with a competition assay format. The choice of which mode or format of immunoaffinity binding assay to use will depend on the intent of the user. Such methods can be found described in common laboratory manuals such as Harlow and Lane, supra, 1999.

As with the hybridization methods described previously, the diagnostic formats employing affinity binding can be used in conjunction with a variety of detection labels and systems known in the art to quantitate mannosyl transferase amounts in the analyzed sample. Detection systems include the detection of bound mannosyl transferase by both direct and indirect means. Direct detection methods include labeling of the mannosyl transferase-selective antibody or binding agent. Indirect detection systems include, for example, the use of labeled secondary antibodies and binding agents.

Secondary antibodies, labels and detection systems are well known in the art and can be obtained commercially or by techniques well known in the art. The detectable labels and systems employed with the mannosyl transferase-selective binding agent should not impair binding of the agent to mannosyl transferase. Moreover, multiple antibody and label systems can be employed for detecting the bound mannosyl transferase-selective antibody to enhance the sensitivity of the binding assay if desired.

As with the hybridization formats described previously, detectable labels can be essentially any label that can be quantitated or measured by analytical methods. Such labels include, for example, enzymes, radioisotopes, fluorochromes as well as chemi- and bioluminescent compounds. Specific examples of enzyme labels include horseradish peroxidase (HRP), alkaline phosphatase (AP), β-galactosidase, urease and luciferase.

A horseradish-peroxidase detection system can be used, for example, with the chromogenic substrate tetramethylbenzidine (TMB), which yields a soluble product in the presence of hydrogen peroxide that is detectable by measuring absorbance at 450 nm. An alkaline phosphatase detection system can be used with the chromogenic substrate p-nitrophenyl phosphate, for example, which yields a soluble product readily detectable by measuring absorbance at 405 nm. Similarly, a β-galactosidase detection system can be used with the chromogenic substrate o-nitrophenyl-β-D-galactopyranoside (ONPG), which yields a soluble product detectable by measuring absorbance at 410 nm, or a urease detection system can be used with a substrate such as urea-bromocresol purple (Sigma Immunochemicals, St. Louis, Mo.). Luciferin is the substrate compound for luciferase which emits light following ATP-dependent oxidation.

Fluorochrome detection labels are rendered detectable through the emission of light of ultraviolet or visible wavelength after excitation by light or another energy source. DAPI, fluorescein, Hoechst 33258, R-phycocyanin, B-phycoerythrin, R-phycoerythrin, rhodamine, Texas red and lissamine are specific examples of fluorochrome detection labels that can be utilized in the affinity binding formats of the invention. Particularly useful fluorochromes is fluorescein or rhodamine.

Chemiluminescent as well as bioluminescent detection labels are convenient for sensitive, non-radioactive detection of mannosyl transferase and can be obtained commercially from various sources such as Amersham Lifesciences, Inc. (Arlington Heights, Ill.).

Radioisotopes can alternatively, be used as detectable labels for use in the mannosyl transferase binding assays of the invention. Iodine-125 is a specific example of a radioisotope useful for a detectable label.

Signals from detectable labels can be analyzed, for example, using a spectrophotometer to detect color from a chromogenic substrate; a fluorometer to detect fluorescence in the presence of light of a certain wavelength; or a radiation counter to detect radiation, such as a gamma counter for detection of iodine-125. For detection of an enzyme-linked secondary antibody, for example, a quantitative analysis of the amount of bound agent can be made using a spectrophotometer such as an EMAX Microplate Reader (Molecular Devices, Menlo Park, Calif.) in accordance with the manufacturer's instructions. If desired, the assays of the invention can be automated or performed robotically, and the signal from multiple samples can be detected simultaneously.

The diagnostic formats of the present invention can be forward, reverse or simultaneous as described in U.S. Pat. No. 4,376,110 and U.S. Pat. No. 4,778,751. Separation steps for the various assay formats described herein, including the removal of unbound secondary antibody, can be performed by methods known in the art (Harlow and Lane, supra, 1999). For example, washing with a suitable buffer can be followed by filtration, aspiration, vacuum or magnetic separation as well as by centrifugation.

Another diagnostic format which can be used for diagnosing or predicting the susceptibility of a bipolar disorder is by measuring the activity of mannsosyl transferase activity in a sample. As with the hybridization and affinity binding formats, activity assays can similarly be performed using essentially identical methods and modes of analysis. Therefore, solution and solid phase modes, including multisample ELISA, RIA and two-dimensional array procedures are applicable for use in measuring the catalytic activity of mannosyl transferase. The activity can be measured by, for example, incubating a mannosyl transferase substrate with the sample and determining the amount of glycosylation. The glycosylation product can be measured using, for example, any of the detectable labels and detection systems described previously. The magnitude of glycoosylation product produced will directly correlate with the mannosyl transferase activity in the sample.

Substrates applicable for determining the activity of mannosyl transferase in a sample include, for example, glycosyl transferase substrates, such as the lipid intermediate dolichol (Dol)-P-Man and GDP-linked mannose. In addition, mannosyl transferase substrate analogs also can be used for determining the amount of mannosyl transferase activity in a sample. Such analogs further can exhibit the capability of releasing a dye or fluorochrome, for example, upon glycosylation by mannosyl tranferase Other glycosylation substrates and analogs of lipid intermediate dolichol (Dol)-P-Man and GDP-linked monosaccharides such as GDP-mannose are well known to those skilled in the art and are similarly applicable in the methods of the invention for determining mannosyl transferase activity in a sample. Once a sample has been contacted with a mannosyl transferase substrate or analog thereof, the activity level of the mannosyl transferase can be measured by a variety of methods known in the art, including substrate conversion, product formation or the presence of an intermediate of a lipid-linked oligosaccharide such as, for example, lipid-linked Man₆GlcNAC₂.

Thus, the invention provides a method of diagnosing or predicting the susceptibility of a bipolar disorder. The method consists of measuring an expression or activity level of mannosyl transferase in a sample obtained from an individual, and comparing the measured expression or activity level of mannosyl transferase to mannosyl transferase expression or activity levels from a reference sample, wherein a reduction of the measured expression or activity levels compared to the reference sample indicates the occurrence or susceptibility of a bipolar disorder.

The invention also provides a method of identifying a compound that modulates the activity a mannosyl transferase by contacting a sample containing a mannosyl transferase having substantially the same sequence as SEQ ID NO: 2, or a functional fragment thereof, and a substrate with a test compound under conditions that allow conversion of substrate to product, and measuring the amount of substrate conversion, a change in the amount of substrate conversion in the presence of said test compound indicating that said compound has mannosyl transferase modulatory activity.

As used herein, the term “modulatory activity” as used in reference to mannosyl transferase, is intended to mean causing an alteration in the amount or activity of a mannosyl transferase polypeptide compared to a reference level of mannosyl transferase amount or activity. Such alterations include an increase or decrease in amount or activity of a mannosyl transferase. The amount or activity of a mannosyl transferase can be modulated by promoting or inhibiting the cellular processes that regulate mannosyl transferase levels or activity.

A compound that has mannosyl transferase modulatory activity can be a molecule that specifically binds a mannosyl transferase nucleic acid or polypeptide and alters its expression or activity. A compound that has mannosyl transferase modulatory activity also can be a naturally occurring macromolecule, such as a peptide, nucleic acid, carbohydrate, lipid, or any combination thereof. A compound that has mannosyl transferase modulatory activity also can be a partially or completely synthetic derivative, analog or mimetic of such a macromolecule, or a small organic or inorganic molecule prepared partly or completely by combinatorial chemistry methods.

The amount or activity of a mannosyl transferase can be modulated in a direct manner, for example, by modification or conformational change of the mannosyl transferase polypeptide that increases or decreases its catalytic activity. A mannosyl transferase also can be modulated indirectly, for example, by stimulating or inhibiting regulators of mannosyl transferase polypeptide expression. Therefore, the term modulating activity encompasses altering the amount or activity of a mannosyl transferase by both direct and indirect methods and includes both increasing and decreasing the activity of mannosyl transferase.

A test compound added to a sample containing mannosyl transferase and a mannosyl transferase substrate which increases or decreases the amount or rate of mannosyl transferase substrate conversion compared to the absence of the test compound indicates that the compound increases mannosyl transferase activity. Therefore, the invention provides a method of identifying compounds that modulate the activity of mannosyl transferase. The mannosyl transferase containing sample used for such a method can be cell fractionate, cell tissue, a cell population or a recombinant cell population expressing mannosyl transferase.

The methods for determining the activity of mannosyl transferase in a sample described above can also be adapted for screening test compounds to determine their ability to inhibit or increase mannosyl transferase mediated glycosylaton of its substrates. In such cases, a test compound is added to a reaction system and the effect of the test compound on production of conversion product is observed. Those compounds which increase the production or rate of cleavage product are considered as potential mannosyl transferase agonists and further as potential therapeutic agents for the treatment of a bipolar disorder.

A reaction system for identifying a compound that inhibits or enhances mannosyl transferase activity can be performed using essentially any source of mannosyl transferase activity. Such sources include, for example, a cell sample such as a lysate or fractionated portion; a recombinant cell or soluble recombinant source, and an in vitro translated source. The mannosyl transferase source is combined with a mannosyl transferase substrate as described above and incubated in the presence or absence of a test modulatory compound. The conversion rate or extent of the mannosyl transferase substrate in the presence of the test compound is compared with that in the absence of the test compound. Those test compounds which provide increase of the glycolsylation activity of at least about 50% are considered to increase mannosyl transferase activity.

Suitable test compounds for the activity modulation assays can be any substance, molecule, compound, mixture of molecules or compounds, or any other composition which is suspected of being capable of enhancing mannosyl transferase activity in vivo or in vitro. The test compounds can be macromolecules, such as biological polymers, including proteins, polysacchrides and nucleic acids. Sources of test compounds which can be screened for mannosyl transferase inhibitory activity include, for example, libraries of peptides, polypeptides, DNA, RNA and small organic compounds. Additionally, test compounds can be preselected based on a variety of criteria. For example, suitable test compounds can be selected as having known glycosylation enhancement activity. Alternatively, the test compounds can be selected randomly and tested by the screening methods of the present invention. Test compounds are administered to the reaction system at a concentration in the range from about 1 nM to 1 mM.

The present invention further provides a transgenic non-human mammal expressing an exogenous nucleic acid encoding substantially the same amino acid sequence as shown as SEQ ID NO: 2. As employed herein, the phrase “exogenous nucleic acid” refers to nucleic acid sequence which is not native to the host, or which is present in the host in other than its native environment, for example, as part of a genetically engineered DNA construct. In addition to naturally occurring levels of a mannosyl transferase, a mannosyl transferase polypeptide of the invention can either be overexpressed or underexpressed in transgenic mammals, for example, underexpressed in a knock-out animal.

Also provided are transgenic non-human mammals capable of expressing nucleic acids encoding mannosyl transferase polypeptides so mutated as to be incapable of normal activity. A transgenic non-human mammal can express, for example, a disrupted mannosyl transferase gene. Therefore, the transgenic non-human mammals do not express native mannosyl transferase or have reduced expression of native mannosyl transferase. The present invention also provides transgenic non-human mammals having a genome comprising antisense nucleic acids complementary to nucleic acids encoding mannosyl transferase polypeptides, placed so as to be transcribed into antisense mRNA complementary to mRNA encoding mannosyl transferase polypeptides, which hybridizes to the mRNA and, thereby, reduces the translation thereof. The nucleic acid can additionally comprise an inducible promoter and/or tissue specific regulatory elements, so that expression can be induced, or restricted to specific cell types.

Animal model systems useful for elucidating the physiological and behavioral roles of mannosyl transferase polypeptides are also provided, and are produced by creating transgenic animals in which the expression of the mannosyl transferase polypeptide is altered using a variety of techniques. Examples of such techniques include the insertion of normal or mutant versions of nucleic acids encoding an mannosyl transferase polypeptide by microinjection, retroviral infection or other means well known to those skilled in the art, into appropriate fertilized embryos to produce a transgenic animal, see, for example, Hogan et al., Manipulating the Mouse Embryo: A Laboratory Manual (Cold Spring Harbor Laboratory, (1986)). Transgenic animal model systems are useful for in vivo screening of compounds for identification of specific ligands, such as agonists or antagonists, which activate or inhibit a biological activity.

Also provided herein, is the use of homologous recombination of mutant or normal versions of mannosyl transferase genes with the native gene locus in transgenic animals, to alter the regulation of expression or the structure of mannosyl transferase polypeptides by replacing the endogeneous gene with a recombinant or mutated mannosyl transferase gene. Methods for producing a transgenic non-human mammal including a gene knock-out non-human mammal, are well known to those skilled in the art (see, Capecchi et al., Science 244:1288 (1989); Zimmer et al., Nature 338:150 (1989); Shastry, Experentia 51:1028-1039 (1995); Shastry, Mol. Cell. Biochem., 181:163-179 (1998); and U.S. Pat. No. 5,616,491, issued Apr. 1, 1997; U.S. Pat. No. 5,750,826, issued May 12, 1998; and U.S. Pat. No. 5,981,830, issued Nov. 9, 1999).

The invention further provides a method of reducing the severity or progression of bipolar disorder comprising administering to a subject an effective amount of a substance that reduces the effects of alterations in proteoglycan synthesis and processing associated with bipolar disorder.

A substance that reduces the effects of alterations in proteoglycan synthesis and processing associated with bipolar disorder can have the enzymatic activity of mannosyl transferase and can be, for example, a mannosyl transferase having substantially the same amino acid sequence as shown as in SEQ ID NO: 2, or a functional fragment thereof. A substance that reduces the effects of alterations in proteoglycan synthesis and processing associated with bipolar disorder can contain substantially the same nucleotide sequence as shown as SEQ ID NO: 1, or a fragment thereof, and can encode substantially the same amino acid sequence as shown as SEQ ID NO: 2, or a functional fragment thereof. Alternatively, a substance that reduces the effects of alterations in proteoglycan synthesis and processing associated with bipolar disorder can be an intermediate in the assembly pathway of a lipid-linked oligosaccaride. In particular, a substance that reduces the effects of alterations in proteoglycan synthesis and processing associated with bipolar disorder can be the intermediate that represents either the product or the substrate of the catalytic conversion responsible for the observed alterations in proteoglycan synthesis and processing.

The invention provides methods for introducing a nucleic acid encoding a mannosyl transferase into a cell in a subject, for example, for gene therapy. Viruses are specialized infectious agents that can elude host defense mechanisms and can infect and propagate in specific cell types. Viral based systems provide the advantage of being able to introduce relatively high levels of a heterologous nucleic acid into a variety of cells. Viral vectors based upon retroviruses, adeno-associated viruses, herpes viruses and adenoviruses are all used as gene therapy vectors for the introduction of therapeutic nucleic acids into the cells of an organism by ex vivo and in vivo methods. Suitable viral vectors for introducing invention nucleic acid encoding an mannosyl transferase polypeptide into mammalian cells are well known in the art. These viral vectors include, for example, Herpes simplex virus vectors (e.g., Geller et al., Science, 241:1667-1669 (1988)), Vaccinia virus vectors (e.g., Piccini et al., Meth. in Enzymology, 153:545-563 (1987); Cytomegalovirus vectors (Mocarski et al., in Viral Vectors, Y. Gluzman and S. H. Hughes, Eds., Cold Spring Harbor Laboratory, Cold Spring Harbor, N.Y., 1988, pp. 78-84), Moloney murine leukemia virus vectors (Danos et al., Proc. Natl. Acad. Sci., USA, 85:6469 (1980)), adenovirus vectors (e.g., Logan et al., Proc. Natl. Acad. Sci., USA, 81:3655-3659 (1984); Jones et al., Cell, 17:683-689 (1979); Berkner, Biotechniques, 6:616-626 (1988); Cotten et al., Proc. Natl. Acad. Sci., USA, 89:6094-6098 (1992); Graham et al., Meth. Mol. Biol., 7:109-127 (1991)), adeno-associated virus vectors, retrovirus vectors (see, e.g., U.S. Pat. Nos. 4,405,712 and 4,650,764), and the like. Viral vectors useful for practicing the methods of the invention such as, for example, adenovirus and retroviral vectors, are well known in art and can be obtained from commercial sources such as Promega (Madison Wis.), Stratagene (La Jolla Calif.) or GIBCO/BRL (Gaithersburg Md.) or can be constructed by one skilled in the art (Ausubel et al., supra, 2000).

Suitable retroviral vectors for use herein also are described, for example, in U.S. Pat. No. 5,252,479, and in WIPO publications WO 92/07573, WO 90/06997, WO 89/05345, WO 92/05266 and WO 92/14829, incorporated herein by reference, which provide a description of methods for efficiently introducing nucleic acids into human cells using such retroviral vectors. Other retroviral vectors include, for example, the mouse mammary tumor virus vectors (e.g., Shackleford et al., Proc. Natl. Acad. Sci. USA, 85:9655-9659 (1988)), and the like.

In particular, the specificity of viral vectors for particular cell types can be utilized to target predetermined cell types. Thus, the selection of a viral vector will depend, in part, on the cell type to be targeted. For example, if a bipolar disorder is to be treated by increasing the level of a mannosyl transferase polypeptide in neuronal cells affected by the disease, then a viral vector that targets neuronal cells can be used. A vector derived from a herpes simplex virus is an example of a viral vector that targets neuronal cells (Battleman et al., J. Neurosci. 13:941-951 (1993), which is incorporated herein by reference). In addition, a viral vector or other vector can be constructed to express a nucleic acid encoding a mannosyl transferase in a tissue specific manner by incorporating a tissue-specific promoter or enhancer into the vector (Dai et al., Proc. Natl. Acad. Sci. USA 89:10892-10895 (1992), which is incorporated herein by reference).

In one embodiment, the invention provides an expression vector that contains a neural tissue-selective regulatory element operatively linked to a nucleic acid molecule encoding a mannosyl transferase polypeptide. A neural tissue-selective regulatory element is a nucleotide sequence that, when operatively linked to a nucleic acid molecule, confers selective expression upon the nucleic acid molecule in a limited number of tissue types, including central or peripheral nervous tissue. Exemplary neural tissue-selective regulatory elements include the Schwann cell-specific partial collagen type VI promoter (Braghetta et al., J. Cell Biol. 135: 1163-1177 (1996)) and regulatory elements derived from the glial fibrillary acidic protein (GFAP) gene, which directs specific expression in astrocytes in the central nervous system in addition to Schwann cell expression (Mucke et al., New Biol. 3:465-474 (1991); Balcarek and Cowan, Nucleic Acids Res. 13:5527-5543 (1985)). Neural tissue-selective regulatory elements also include elements derived from the neuron specific enolase gene, such as the 5′ 1.8 kb SacI-XhoI fragment of the rat neuron-specific enolase gene which has been shown to direct neuronal expression (Forss-Petter et al., Neuron 5:187-197 (1990)). A neural tissue-specific regulatory element also can be, for example, a myelin basic protein (MBP) promoter or a functional fragment thereof, such as the 1.3 kb HindIII fragment of murine myelin basic protein which directs oligodendrocyte expression (Okano et al., EMBO J. 7:77-83 (1988); Tamura et al., Nucleic Acids Res. 16:11441-11459 (1988); Turnley et al., Nature 353:566-569 (1991); see, also, Campbell, Brain Research Reviews 26:327-336 (1998)).

For gene therapy, a vector containing a nucleic acid encoding a mannosyl transferase or an antisense nucleotide sequence can be administered to a subject by various methods. For example, if viral vectors are used, administration can take advantage of the target specificity of the vectors. In such cases, there is no need to administer the vector locally at the diseased site. However, local administration can be a particularly effective method of administering a nucleic acid encoding a mannosyl transferase. In addition, administration can be via intravenous or subcutaneous injection into the subject. Following injection, the viral vectors will circulate until they recognize host cells with the appropriate target specificity for infection. Injection of viral vectors into the spinal fluid also can be an effective mode of administration, for example, in treating a bipolar disorder.

Receptor-mediated DNA delivery approaches also can be used to deliver a nucleic acid molecule encoding a mannosyl transferase into cells in a tissue-specific manner using a tissue-specific ligand or an antibody that is non-covalently complexed with the nucleic acid molecule via a bridging molecule (Curiel et al., Hum. Gene Ther. 3:147-154 (1992); Wu and Wu, J. Biol. Chem. 262:4429-4432 (1987), each of which is incorporated herein by reference). Direct injection of a naked or a nucleic acid molecule encapsulated, for example, in cationic liposomes also can be used for stable gene transfer into non-dividing or dividing cells in vivo (Ulmer et al., Science 259:1745-1748 (1993), which is incorporated herein by reference). In addition, a nucleic acid encoding a mannosyl transferase can be transferred into a variety of tissues using the particle bombardment method (Williams et al., Proc. Natl. Acad. Sci. USA 88:2726-2730 (1991), which is incorporated herein by reference). Such nucleic acids can be linked to the appropriate nucleotide sequences required for transcription and translation.

A particularly useful mode of administration of a nucleic acid encoding a mannosyl transferase is by local administration. Local administration can be advantageous because there is no dilution effect and, therefore, the likelihood that a majority of the targeted cells will be contacted with the nucleic acid molecule is increased. Thus, local inoculation can alleviate the targeting requirement necessary with other forms of administration and, if desired, a vector that infects all cell types in the inoculated area can be used. If expression is desired in only a specific subset of cells within the inoculated area, then a promoter, an enhancer or other expression element specific for the desired subset of cells can be linked to the nucleic acid molecule. Vectors containing such nucleic acid molecules and regulatory elements can be viral vectors, viral genomes, plasmids, phagemids and the like. Transfection vehicles such as liposomes also can be used to introduce a non-viral vector into recipient cells. Such vehicles are well known in the art.

Therefore, the encoding nucleic acids for the mannosyl transferase polypeptides of the invention are useful in conjunction with a wide variety of gene therapy methods known in the art for delivering an effective amount of the polypeptide, or functional fragment thereof. Using the teachings and guidance provided herein, the encoding nucleic acid for a mannosyl transferase polyptpide, or a functional fragment thereof, can be incorporated into a vector or delivery system known in the art and used for delivery and expression of the encoding sequence to achieve a therapeutic effect. As described above, applicable vector and delivery systems known in the art include, for example, retroviral vectors, adenovirus vectors, adeno-associated virus, ligand conjugated particles and nucleic acids for targeting, isolated DNA and RNA, liposomes, polylysine, and cell therapy, employing the transplantation of cells modified to express mannosyl transferase polypeptides, or functional fragments therof, as well as various other gene delivery methods and modifications known to those skilled in the art, such as those described in Shea et al., Nature Biotechnol. 17:551-554 (1999), which is incorporated herein by reference.

The present invention also provides therapeutic compositions useful for practicing the therapeutic methods described herein. Therapeutic compositions of the present invention, such as pharmaceutical compositions, contain a physiologically compatible carrier together with a mannosyl transferase modulating agent, a mannosyl transferase polypeptide of the invention, or functional fragment thereof, such as a compound (agonist or antagonist) identified by the methods described herein, or an anti-mannosyl transferase antibody, as described herein, dissolved or dispersed therein as an active ingredient. In a preferred embodiment, the therapeutic composition is not immunogenic when administered to a mammal or human patient for therapeutic purposes.

The preparation of a pharmacological composition that contains active ingredients dissolved or dispersed therein is well known in the art. A pharmaceutically acceptable composition containing a mannosyl transferase polypeptide of the invention is capable of administration to a mammal without the production of undesirable physiological effects such as nausea, dizziness, gastric upset, and the like. Typically such compositions are prepared as injectables either as liquid solutions or suspensions; however, solid forms suitable for solution, or suspension, in liquid prior to use can also be prepared. The preparation also can be emulsified.

The active ingredient can be mixed with excipients which are pharmaceutically acceptable and compatible with the active ingredient in amounts suitable for use in the therapeutic methods described herein. Suitable excipients are, for example, water, saline, dextrose, glycerol, ethanol, or the like, as well as combinations of any two or more thereof. In addition, if desired, the composition can contain minor amounts of auxiliary substances such as wetting or emulsifying agents, pH buffering agents, and the like, which enhance the effectiveness of the active ingredient.

The therapeutic composition of the present invention can include pharmaceutically acceptable salts of the components therein. Pharmaceutically acceptable nontoxic salts include the acid addition salts formed with the free amino groups of the polypeptide that are formed with inorganic acids such as, for example, hydrochloric acid, hydrobromic acid, perchloric acid, nitric acid, thiocyanic acid, sulfuric acid, phosphoric acid, acetic acid, propionic acid, glycolic acid, lactic acid, pyruvic acid, oxalic acid, malonic acid, succinic acid, maleic acid, fumaric acid, anthranilic acid, cinnamic acid, naphthalene sulfonic acid, sulfanilic acid, and the like.

Salts formed with the free carboxyl groups also can be derived from inorganic bases such as, for example, sodium hydroxide, ammonium hydroxide, potassium hydroxide, and the like; and organic bases such as mono-, di-, and tri-alkyl and -aryl amines, for example, triethylamine, diisopropyl amine, methyl amine, dimethyl amine, and the like, and optionally substituted ethanolamines, for example, ethanolamine, diethanolamine, and the like.

Physiologically tolerable carriers are well known in the art. Exemplary liquid carriers are sterile aqueous solutions that contain no materials in addition to the active ingredients and water, or contain a buffer such as sodium phosphate at physiological pH, physiological saline or both, such as phosphate-buffered saline. Still further, aqueous carriers can contain more than one buffer salt, as well as salts such as sodium and potassium chlorides, dextrose, polyethylene glycol and other solutes.

Liquid compositions can also contain liquid phases in addition to and to the exclusion of water. Exemplary additional liquid phases include glycerin, vegetable oils such as cottonseed oil, and water-oil emulsions.

As described herein, an effective amount is a predetermined amount calculated to achieve the desired therapeutic effect, for example, to modulate the catalytic activity of a mannosyl transferase polypeptide of the invention. The required dosage will vary with the particular treatment and with the duration of desired treatment; however, it is anticipated that dosages between about 10 micrograms and about 1 milligram per kilogram of body weight per day will be used for therapeutic treatment. It may be particularly advantageous to administer such compounds in depot or long-lasting form as discussed hereinafter. A therapeutically effective amount is typically an amount of a mannosyl transferase polypeptide, mannosyl transferase modulating agent or other compound identified herein that, when administered in a physiologically acceptable composition, is sufficient to achieve a plasma concentration of from about 0.1 μg/ml to about 100 μg/ml, or from about 1.0 μg/ml to about 50 μg/ml, or alternatively at least about 2 μg/ml and usually 5 to 10 μg/ml.

A substance that reduces the effects of alterations in proteoglycan synthesis and processing associated with bipolar disorder can be administered as a solution or suspension together with a pharmaceutically acceptable medium. Such a pharmaceutically acceptable medium can be, for example, a sterile aqueous solvent such as sodium phosphate buffer, phosphate buffered saline, normal saline or Ringer's solution or other physiologically buffered saline, or other solvent or vehicle such as a glycol, glycerol, an oil such as olive oil or an injectable organic ester. A pharmaceutically acceptable medium can additionally contain physiologically acceptable compounds that act, for example, stabilize the substance that reduces the effects of alterations in proteoglycan synthesis and processing associated with bipolar disorder, increase its solubility, or increase its absorption. Such physiologically acceptable compounds include, for example, carbohydrates such as glucose, sucrose or dextrans; antioxidants such as ascorbic acid or glutathione; receptor mediated permeabilizers, which can be used to increase permeability of the blood-brain barrier; chelating agents such as EDTA, which disrupts microbial membranes; divalent metal ions such as calcium or magnesium; low molecular weight proteins; lipids or liposomes; or other stabilizers or excipients. Those skilled in the art understand that the choice of a pharmaceutically acceptable carrier depends on the route of administration of the compound containing the substance that reduces the effects of alterations in proteoglycan synthesis and processing associated with bipolar disorder and on its particular physical and chemical characteristics.

Formulations suitable for parenteral administration include aqueous and non-aqueous sterile injection solutions such as the pharmaceutically acceptable mediums described above. The solutions can additionally contain, for example, buffers, bacteriostats and solutes which render the formulation isotonic with the blood of the intended recipient. Other formulations include, for example, aqueous and non-aqueous sterile suspensions which can include suspending agents and thickening agents. The formulations can be presented in unit-dose or multi-dose containers, for example, sealed ampules and vials, and can be stored in a lyophilized condition requiring, for example, the addition of the sterile liquid carrier, immediately prior to use. Extemporaneous injection solutions and suspensions can be prepared from sterile powders, granules and tablets of the kind previously described.

For applications that require the compounds and compositions to cross the blood-brain barrier, formulations that increase the lipophilicity of the compound are particularly desirable. For example, a substance that reduces the effects of alterations in proteoglycan synthesis and processing associated with bipolar disorder can be incorporated into liposomes (Gregoriadis, Liposome Technology, Vols. I to III, 2nd ed. (CRC Press, Boca Raton Fla. (1993)). Liposomes, which consist of phospholipids or other lipids, are nontoxic, physiologically acceptable and metabolizable carriers that are relatively simple to make and administer.

A substance that reduces the effects of alterations in proteoglycan synthesis and processing associated with bipolar disorder also can be prepared as nanoparticles. Adsorbing peptide compounds onto the surface of nanoparticles has proven effective in delivering peptide drugs to the brain (see Kreuter et al., Brain Res. 674:171-174 (1995)). Exemplary nanoparticles are colloidal polymer particles of poly-butylcyanoacrylate with a substance that reduces the effects of alterations in proteoglycan synthesis and processing associated with bipolar disorder adsorbed onto the surface and then coated with polysorbate 80.

Image-guided ultrasound delivery of a substance capable of reducing the effects of alterations in proteoglycan synthesis and processing associated with bipolar disorder through the blood-brain barrier to selected locations in the brain can be utilized as described in U.S. Pat. No. 5,752,515. Briefly, to deliver a substance that reduces the effects of alterations in proteoglycan synthesis and processing associated with bipolar disorder past the blood-brain barrier a selected location in the brain is targeted and ultrasound used to induce a change detectable by imaging in the central nervous system (CNS) tissues and/or fluids at that location. At least a portion of the brain in the vicinity of the selected location is imaged, for example, via magnetic resonance imaging (MRI), to confirm the location of the change. A substance that reduces the effects of alterations in proteoglycan synthesis and processing associated with bipolar disorder can delivered to the confirmed location by applying ultrasound to effect opening of the blood-brain barrier at that location and, thereby, to induce uptake of the substance.

In addition, polypeptides called receptor mediated permeabilizers (RMP) can be used to increase the permeability of the blood-brain barrier to molecules such as therapeutic agents or diagnostic agents as described in U.S. Pat. Nos. 5,268,164; 5,506,206; and 5,686,416. These receptor mediated permeabilizers can be intravenously co-administered to a host with molecules whose desired destination is the cerebrospinal fluid compartment of the brain. The permeabilizer polypeptides or conformational analogues thereof allow therapeutic agents to penetrate the blood-brain barrier and arrive at their target destination.

In current treatment regimes for bipolar disorder, more than one compound often is administered to an individual for management of the same or different aspects of the disease. Similarly, in the methods of the invention involving reducing the effects of alterations in proteoglycan synthesis and processing associated with bipolar disorder, a substance can advantageously be formulated with a second therapeutic compound such as an mood stabilizer, anticonvulsant compound, antipsychotic compound or any other compound that manages the same or different aspects of the disease. Such compounds include, for example, lithium, divalproex, lamotrigine, carbamazepine, gabapentin. Contemplated methods of reducing the severity of a bipolar disorder include administering a substance that reduces the effects of alterations in proteoglycan synthesis and processing associated with bipolar disorder alone, in combination with, or in sequence with, such other compounds. Alternatively, combination therapies can consist of fusion proteins, where the substance that reduces the effects of alterations in proteoglycan synthesis and processing associated with bipolar disorder is linked to a heterologous protein, such as a therapeutic protein.

It is understood that modifications which do not substantially affect the activity of the various embodiments of this invention are also included within the definition of the invention provided herein. Accordingly, the following examples are intended to illustrate but not limit the present invention.

### EXAMPLE I

### Isolation and Characterization of a Chromosomal Breakpoint Associated with a Balanced Translocation t(9;11)(p24;q23.1)

This example describes the isolation and characterization of a chromosomal breakpoint on human chromosome 11 that is associated with a balanced translocation t(9;11)(p24;q23.1), which co-segregates with bipolar mood disorder.

A pedigree with a segregating t(9;11) chromosomal translocation that has been previously reported to segregate with bipolar affective disorder in Smith et al., Am. J. Hum. Genet. 45:A178 (1989), was selected for molecular analysis. In this family, all affected individuals have the balanced translocation, five of 11 translocation carriers have bipolar illness, and one carrier suffers from unipolar illness.

In order to isolate the chromosomal breakpoint and define the genes in the region of the breakpoint, analysis of detailed physical maps was combined with large scale DNA sequencing. The physical map was constructed generally as described by Quackenbush et al., Genomics 29:512-525 (1995), which is incorporated herein by reference, with sequence tag sites (STS) probes and PCR conditions as described in Smith et al., Genomics 17:699-725 (1993), which also is incorporated herein by reference. Human/rodent somatic cell hybrids containing a normal chromosome 9 (PK87-9), chromosome 11 (Jl), derivative t(9; 11) chromosomes (EN311, EN308 EN328 and EN342) were utilized for mapping studies and the isolation of RNA for expression analysis as set forth below.

YAC clones were isolated from a yeast artificial chromosome library prepared at the Centre d'etude du Polymorhisme Humain (CEPH) and from a chromosome 11-specific library from Roswell Park Cancer Institute as described in Qin et al., Proc. Natl. Acad. Sci. USA 93:3149-3154 (1996), which is incorporated herein by reference. Two YAC clones, pRP 18g8 (350 kb) and pRP12a6 (100 kb) that span this breakpoint on chromosome 11, as well as five cosmid clones were identified. Probes derived from the ends of one cosmid clone NB1H10 detected one novel rearrangement in multiple digests of genomic DNA from a carrier individual by Southern blots. Digestion with EcoR1 localized the breakpoint to within a 4.4 kb restriction fragment.

To determine if the breakpoint was contained within NB1H10, a probe was derived from the T7-end of the cosmid clone and analyzed using somatic cell hybrids derived from t(9;11) cell lines as described in Richard and Washington, Construction and Assay of Radiation Hybrids in “Current Protocols in Human Genetics,” John Wiley & Sons Publ., New York, which is incorporated herein by reference. These results suggested that NB1H10 was located centromeric to the chromosomal breakpoint on chromosome 11 and, further, predicted a 20 kb gap between the T7 end of NB1H10 and cosmid NBRE2. PCR probes were derived from end sequences of YAC pRP12a6 12a6 spanning the breakpoint and used to screen a human PAC library representing 10-fold coverage of the human genome. Thirty-two clones were detected spanning the breakpoint. End-sequencing and production of additional PCR probes, as well as the analysis of somatic cell hybrids, established a physical map of this region and mapped the breakpoint to within PAC pDJl5901. To identify genes potentially affected by the translocation on chromosome 11, PAC clone pDJl5901 was sequenced by M13 shotgun cloning followed by automated fluorescence-based sequencing using a mixture of dye-primer and dye-terminator chemistries. Sequence tracings were determined using AB1377 automated gene sequencers and contigs assembled using Phred/Phrap assembly programs. A 6× shotgun coverage was determined followed by selective gap filling using synthetic oligonucleotides produced using specialized software and hardware. The sequence error rate was estimated from Phred/Phrap scores as no<1 error in 10-4 nucleotides. The resulting sequence spanned 106 kb and was found to contain end-sequences from cosmid and PAC clones, orienting the physical map.

DNA sequence analysis revealed an open reading frame encoding a novel protein, pmt. DNA sequence analysis was done using Phred and Phrap for assembly of sequencing information, and Autosearch and Autofeatures A for automated annotation and analysis. Vector-NTI, AlignX and BioPlot (InfoMax) were used for protein sequence comparisons as well as the Blast server at the National Center for Biotechnology Information and the AutoSearch automated search engine at University of Texas, Southwestern Medical Center, which revealed no known human gene similarity, but a marked similarity to a gene derived from the complete genomic sequence of yeast S. cerevisiae.

The translated sequence revealed a putative protein product of 611 amino acid residues, consisting of seven hydrophobic transmembrane domains and a carboxy terminal hydrophilic domain. The sequence shows approximately 46.9% identity with the ALG9 gene of S. cerevisia (p value less than 1×10−138). The sequence also detected seven human Expressed Sequence Tags (ESTs) with p values ranging from 2×10−20 to 2.3×10−155. This analysis establishes human mannosyl transferase as a human homolog of yeast ALG9.

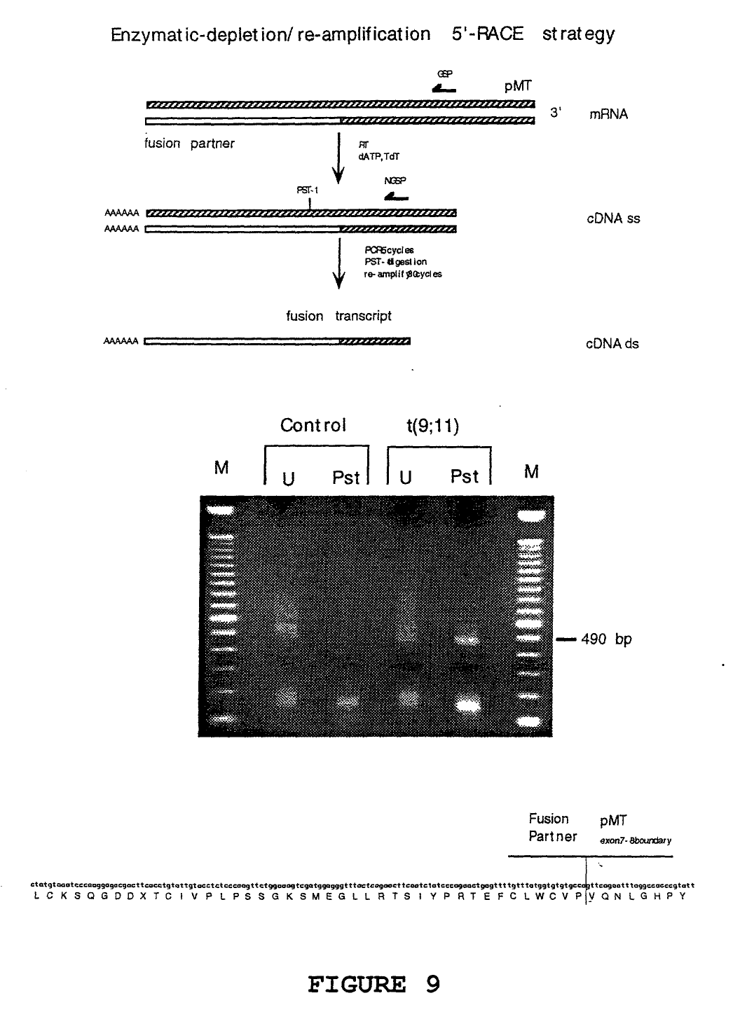

Mapping analysis revealed that the chromosome translocation occurred within the mannosyl transferase gene resulting in fusion partners between exons 6 and 8 (FIG. 3). To determine the nature of the fusion, and the presence of additional genes on chromosome 9, probes derived from the chromosome 11 physical map were utilized to screen libraries constructed from a somatic cell hybrid containing the 9;11 derivative chromosome. Cosmid clones were sequenced and RT-PCR carried out to establish the sequence of a fusion transcript. PACE PCR for extending transcripts was performed as previously described in Frohman et al., Proc. Nat. Acad. Sci. USA 85:8998 (1988) and Loh, Science 243:217-220 (1989), both of which are incorporated herein by reference. As ilustrated in FIG. 9, 5′ RACE amplification was used to determine the sequence upstream (5′) of the chromosome 9;11 translocation breakpoint. DNA was isolated from a cell line carrying the translocation and a amplification oligonucleotide primer was synthesized corresponding to the distal region of mannosyl transferase sequence at the exon 7 exon 8 boundary. 5′ RACE amplification was carried out to extend the amplified DNA sequence into the 5′ region of the putative fusion transcript. Following cDNA synthesis, DNA sequencing was carried out using specific DNA primers and an ABI 377 automated DNA sequencer. The panel in FIG. 9 shows the amplification of a 490 bp fragment from the t(9;11) fusion chromosome indicating amplification of the chromosomal breakpoint.

FIG. 3 shows the resulting fusion transcript present in the carriers of t(9;11). The fusion transcript contains an open reading frame with a 5′ coding sequence from a gene located on chromosome 9 and 3′ coding sequence corresponding to a human mannosyl transferase gene located on chromosome 11 and designated HALG based on its homology to a yeast mannosyl transferase, ALG9.

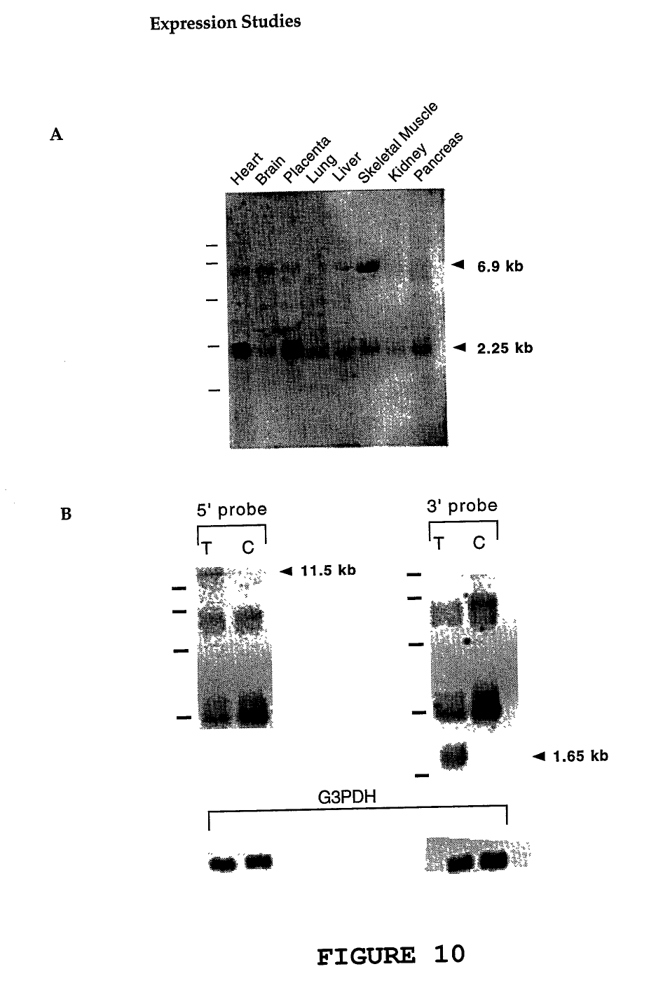

As shown in FIG. 10, Northern blot analysis was carried out using RNA isolated from a variety of normal tissues and from a transformed lymphoblastoid cell line derived from a patient carrying the t(9;11) derivative chromosome. Northern blot analysis was carried out using a fragment of the mannosyl transferase clone carrying the putative human chromosome 11 transcript, and 5′ and 3′ end probes prepared from this clone. Hybridization to Northern blots carrying RNA from a variety of tissues was carried out demonstrating 6.9 kb and 2.25 kb transcripts in most tissues analyzed including heart, brain , placenta, lung, liver, skeletal muscle kidney and pancreas. This demonstrates the wide spread expression pattern of this transcript in most tissues. Hybridization of RNA isolated from control and translocation carrying cell lines demonstrates the presence of a new 1.65 kb transcript with the 3′ mannosyl transferase probe. This demonstrates the presence of a derivative transcript in the translocated, but not the normal cell line, confirming the presence of a novel mRNA. As shown in FIG. 10, hybridization of the same Northern blot with a probe for glucose 3 phosphate dehydrogenase (G3PDH) demonstrated that equivalent amounts of RNA were loaded on lanes of the gel, controlling for selective RNA loss or degradation.

Throughout this application various publications have been referenced. The disclosures of these publications in their entireties are hereby incorporated by reference in this application in order to more fully describe the state of the art to which this invention pertains.

Although the invention has been described with reference to the disclosed embodiments, those skilled in the art will readily appreciate that the specific experiments detailed are only illustrative of the invention. It should be understood that various modifications can be made without departing from the spirit of the invention. Accordingly, the invention is limited only by the following claims.

What is claimed is:

1. An isolated polypeptide, comprising a human mannosyl transferase having substantially the same amino acid sequence as shown as in SEQ ID NO: 2, or a functional fragment thereof.

2. The isolated polypeptide of claim 1, wherein said functional fragment further comprises a substrate binding domain.

3. An isolated nucleic acid, comprising substantially the same nucleotide sequence as shown as SEQ ID NO: 1, or a fragment thereof.

4. The isolated nucleic acid of claim 3, further comprising encoding substantially the same amino acid sequence as shown as SEQ ID NO: 2, or a functional fragment thereof.

5. The isolated nucleic acid of claim 3, wherein said fragment encodes a mannosyl transferase substrate binding domain.

6. The isolated nucleic acid of claim 3, wherein said fragment further comprises an about 15 nucleotide region sequence having substantially the same nucleotide sequence as SEQ ID NO: 1.

7. An isolated polypeptide, comprising a human mannosyl transferase having substantially the same amino acid sequence as shown as in SEQ ID NO: 4, or a functional fragment thereof.

8. An isolated nucleic acid, comprising substantially the same nucleotide sequence as SEQ ID NO: 3, or a fragment thereof.

9. The isolated nucleotide sequence of claim 8, wherein said fragment comprises a 5′ nucleic acid region from human chromosome 9 and a 3′ nucleic acid region from human chromosome 11.

10. An isolated nucleic acid, comprising a human mannosyl transferase encoding nucleic acid and a single nucleotide polymorphism.

11. The isolated nucleic acid of claim 10, wherein said single nucleotide polymorphism is selected from the group consisting of SEQ ID NOS: 16 to 117.

12. A method of diagnosing or predicting the susceptibility of a bipolar disorder, comprising:
(a) contacting a sample obtained from an individual with a probe selective for an altered mannosyl transferase sequence, and
(b) detecting binding of said probe to an analyte in said sample, wherein specific binding of said probe indicates the presence of an altered mannosyl transferase and occurrence or susceptibility of a bipolar disorder.

13. The method of claim 12, wherein said probe comprises substantially the same nucleotide sequence as shown as SEQ ID NO: 1, or a fragment thereof.

14. The method of claim 12, wherein said probe comprises substantially the same nucleotide sequence as shown as SEQ ID NO: 3, or a fragment thereof.

15. The method of claim 12, wherein said probe comprises substantially the same nucleotide sequence as shown as SEQ ID NO: 5, or a fragment thereof.

16. The method of claim 12, wherein said probe comprises a binding agent having specific binding activity to a polypeptide encoded by substantially the same nucleotide sequence as shown as SEQ ID NO: 3, or a fragment thereof.

17. The method of claim 16, wherein said binding agent is an antibody.

18. A method of diagnosing or predicting the susceptibility of a bipolar disorder, comprising:
(a) measuring an expression or activity level of mannosyl transferase in a sample obtained from an individual, and
(b) comparing said measured expression or activity level of mannosyl transferase to mannosyl transferase expression or activity levels from a reference sample, wherein a reduction of said measured expression or activity levels compared to said reference sample indicates the occurrence or susceptibility of a bipolar disorder.

19. The method of claim 18, further comprising contacting said sample with a probe comprising substantially the same nucleotide sequence as shown as SEQ ID NO: 1, or a fragment thereof, wherein said contacting is effected under selective hybridization conditions.

20. The method of claim 18, further comprising contacting said sample with a probe comprising substantially the same nucleotide sequence as shown as SEQ ID NO: 3, or a fragment thereof, wherein said contacting is effected under selective hybridization conditions.

21. The method of claim 18, further comprising contacting said sample with a probe comprising substantially the same nucleotide sequence as shown as SEQ ID NO: 5, or a fragment thereof, wherein said contacting is effected under selective hybridization conditions.

22. The method of claim 18, further comprising contacting said sample with a binding agent having specific binding activity to a human mannosyl transferase having substantially the same amino acid sequence as shown in SEQ ID NO: 2, or a functional fragment thereof, and detecting the presence of specific binding of said binding agent.

23. The method of claim 22, wherein said binding agent is an antibody.

24. The method of claim 22, wherein said binding agent is a substrate analog inhibitor.

25. The method of claim 18, wherein said activity is measured by substrate conversion.

26. The method of claim 18, wherein said activity is measured by product formation.

27. The method of claim 18, further comprising measuring the presence of an intermediate of a lipid-linked oligosaccharide.

28. The method of claim 27, wherein said intermediate is lipid-linked Man₆GlcNAC₂.

29. The method of claim 18, wherein the expression or activity level of mannosyl transferase is measured be determining the presence of hypoglycosylated secretory proteins.

30. The method of claim 29, wherein said hypoglycosylated secretory proteins are characterized by a reduction in N-linked glycosylation.

31. A method of identifying a compound that modulates the activity a mannosyl transferase, comprising:
(a) contacting a sample containing a mannosyl transferase having substantially the same sequence as SEQ ID NO: 2, or a functional fragment thereof, and a substrate with a test compound under conditions that allow conversion of substrate to product, and
(b) measuring the amount of substrate conversion, a change in the amount of substrate conversion in the presence of said test compound indicating that said compound has mannosyl transferase modulatory activity.

32. The method of claim 31, wherein said compound increases the activity of mannosyl transferase.

33. A transgenic non-human mammal expressing an exogenous nucleic acid encoding substantially the same amino acid sequence as shown as SEQ ID NO: 2.

34. The transgenic non-human mammal of claim 33, further comprising a disrupted mannosyl transferase gene.

35. The transgenic non-human mammal of claim 33, wherein said transgenic non-human mammal is a mouse.
